(19) United States
(12) Patent Application Publication
(10) Pub. No.: US 2005/0233305 A1
(43) Pub. Date: Oct. 20, 2005

(54) Sugar chain library constructed via cargo receptor gene modification

(75) Inventors: Kazuo Yamamoto, Bunkyo-ku, Tokyo (JP); Akira Sato, Tottori (JP); Junko Shimauchi, Tokyo (JP); Mariko Matsumoto, Kanagawa (JP)

Correspondence Address: FOLEY AND LARDNER;SUITE 500, 3000 K STREET NW, WASHINGTON, DC, 20007, US

(73) Assignees: Kazuo Yamamoto, Tokyo (JP); Sumitomo Corporation, Tokyo (JP)

(21) Appl. No.: 10/525,020

(22) PCT Filed: Feb. 18, 2003
(86) PCT No.: PCT/JP03/01718

§ 371 Date: Feb. 18, 2005

(30) Foreign Application Priority Data
Aug. 19, 2002 (JP) 2002-238559

(51) Int. Cl. C12Q 1/00; C07H 21/04; C07K 14/71; C12N 15/09

(52) U.S. Cl. 435/4; 435/69.1; 435/320.1; 435/325; 530/350; 536/23.5; 530/395

(57) ABSTRACT

The present invention relates to a method for modifying a carbohydrate moiety of a glycoprotein and a glycoprotein having a modified carbohydrate moiety. The present invention further relates to a cell expressing a glycoprotein with a modified carbohydrate moiety and a method for preparing the same. The present invention further relates to a method for producing a glycoprotein with a modified carbohydrate moiety.

### TECHNICAL FIELD

The present invention relates to a method for modifying a carbohydrate moiety of a glycoprotein and for preparing a glycoprotein having a modified carbohydrate moiety. The present invention further relates to a cell expressing a glycoprotein having a modified carbohydrate moiety and a method for preparing the cell. The present invention further relates to a method for producing a glycoprotein having a modified carbohydrate moiety.

### BACKGROUND ART

Today the entire nucleotide sequences of the human genome is being elucidated and tailor-maid medical treatment is going to begin based on the large amounts of such information. A new era is coming where diagnosis methods for extensively examining protein expression using DNA chips and prescription of drugs based on the data can be carried out via computers. In addition, it is also considered that recombinant proteins will be more easily produced and the use of various cytokines or hormones as pharmaceutical drugs will be increasingly accelerated.

There are recognition mechanisms mediated by various oligosaccharides (sugar chains) in vivo. Since sugar moieties (carbohydrate moieties) attached on proteins have several effects on the transport or the metabolism of glycoproteins, they have great influence on the biological activities of these proteins especially in vivo. Furthermore, it has been suggested that structural changes of sugar moieties of glycoproteins involved in abnormal growth or metastasis of cancer cells. Hence, it is considered that when various proteins are used as drugs, technology for producing various glycoforms of glycoproteins with distinct carbohydrate structures using genetic engineering techniques can serve as technology introducing new drug efficacy. For example, by using such technology, it becomes possible not only to regulate and/or determine cellular localization of glycoproteins, but also to regulate the metabolism of glycoproteins.

However, information concerning about carbohydrate structures of glycoproteins is not directly encoded in genes, so that technology controlling glycosylation cannot be achieved by genome studies or proteome studies directly.

Technology for specifying and controlling carbohydrate structures of glycoproteins has not been established yet. Now carbohydrates attached on proteins are synthesized by sequencial action of various glycosyltransferases (e.g., see JP Patent Publication (Kokai) No. 11-42096 A (1999)). In this procedure, it takes 2 to 3 months for synthesizing oligosaccharides by using manual operation, and a few days are required even when a synthesizer is used. However, recombinant proteins expressed in E. coli have not been glycosylated and moreover, in the case of yeast, carbohydrate moieties on glycoproteins are different from those of humans. Based on this reason, it is difficult to obtain useful recombinant glycoproteins as pharmaceutical drugs.

Recently, needs for oligosaccharides as pharmaceutical drugs is increasing in the field of medicine. For example, a disease such as the congenital disorder of glycosylation type Ib that is an infant metabolic disease is treated by administering oligosaccharides deficient in such patients (Alper, J., Science, 291: 2339, 2001). However, synthesis of oligosaccharides is difficult technically because of the reasons mentioned above and also oligosaccharides are not available because these are very expensive.

Therefore, techniques for producing various kinds of oligosaccharide and for convenient and rapid screening method for useful glycoproteins have been desired.

### SUMMARY OF THE INVENTION

Objects of the present invention are to provide an easy and rapid method for changing carbohydrate moieties of glycoproteins and also a method for producing oligosaccharides from thus obtained glycoproteins. Further object of the present invention is to provide a method for preparing a cell expressing a glycoprotein with different glycoform and for producing an carbohydrate library (sugar chain library).

As a result of intensive studies to achieve the above objects, the present inventors have focused on the fact that structures of carbohydrate moieties of glycoproteins are recognized during sorting process of newly synthesized glycoproteins within the cells. And, they found that carbohydrate structures attached on glycoproteins can be changed and controlled by altering the carbohydrate-binding specificities of cargo receptors that are involved especially in this sorting process. Thus, the present inventors have completed the present invention.

The present invention relates to a method for modifying a carbohydrate moiety of a glycoprotein, which comprises altering a carbohydrate recognition domain of a cargo receptor to modify a carbohydrate moiety of a glycoprotein. Specifically, the method for modifying a carbohydrate moiety of a glycoprotein comprises the following steps of:
(a) changing a nucleotide sequence encoding a carbohydrate recognition domain of a cargo receptor gene;
(b) introducing the above cargo receptor gene into a cell; and
(c) expressing a glycoprotein having a modified carbohydrate moiety in the above cell.

In the above method for modifying a carbohydrate moiety of a glycoprotein, the cargo receptor is preferably VIP36 and/or ERGIC-53.

Furthermore, examples of the glycoprotein include membrane-bound proteins and secretory proteins.

The present invention also relates to a glycoprotein, wherein a carbohydrate moiety is modified by the above method for modifying a carbohydrate moiety.

The present invention also relates to a method for producing a modified oligosaccharide, which comprises cleaving an oligosaccharide from the above glycoprotein.

Furthermore, the present invention relates to a modified oligosaccharide, which is produced by the above production method.

The present invention also relates to a method for preparing a cell expressing a glycoprotein with a modified carbohydrate moiety, which comprises altering a carbohydrate recognition domain of a cargo receptor to modify a carbohydrate moiety of a glycoprotein. Specifically, the preparation method comprises the following steps of:
(a) changing a nucleotide sequence encoding a carbohydrate recognition domain of a cargo receptor gene;
(b) introducing the above cargo receptor gene into a cell;
(c) expressing the above cargo receptor gene in the above cell; and
(d) selecting the cell expressing a glycoprotein having a modified carbohydrate moiety.

Furthermore, the above preparation method may comprise the following steps (e) or (f) (where, in this case, the order of the steps is not specifically limited) of:
(e) introducing the gene of a desired protein into a cell; and
(f) obtaining a cell expressing a desired glycoprotein having a modified carbohydrate moiety.

In the above preparation method, the cargo receptor is preferably VIP36 and/or ERGIC-53.

In addition, examples of the glycoprotein include a membrane-bound protein and a secretory protein.

The present invention also relates to a cell expressing a glycoprotein with a modified carbohydrate moiety, which is prepared by the above preparation method.

Furthermore, the present invention relates to a method for producing a glycoprotein with a modified carbohydrate moiety, which comprises culturing the above cell and collecting a glycoprotein with a modified carbohydrate moiety from the obtained culture.

Furthermore, the present invention relates to a method for constructing a modified carbohydrate library, which comprises introducing random mutations into a carbohydrate recognition domain of a cargo receptor gene and expressing a glycoprotein having a modified carbohydrate moiety. Specifically, the preparation method comprises the following steps of:
(a) introducing random mutations into a nucleotide sequence encoding a carbohydrate recognition domain of a cargo receptor gene;
(b) introducing the cargo receptor gene having the above random mutations into each of a plurality of cells; and
(c) expressing the above cargo receptor gene having random mutations introduced therein in each of the above plurality of cells.

Furthermore, the present invention relates to a method for screening for a test substance interacting with a specific carbohydrate moiety of a glycoprotein or an oligosaccharide cleaved from a glycoprotein or a specific oligosaccharide using the above cell expressing a glycoprotein having a modified carbohydrate moiety. Specifically, the screening method comprises the following steps of:
(a) bringing a cell expressing a glycoprotein having a modified carbohydrate moiety into contact with a test substance; and
(b) determining the interaction between the glycoprotein or the carbohydrate moiety with the test substance.

The above screening method may also comprise a step of growing the cells expressing the glycoprotein with a modified carbohydrate moiety. Furthermore, the above screening method may comprise a step of screening for a cell expressing a desired glycoprotein with a modified carbohydrate moiety or a desired carbohydrate moiety (oligosaccharide) on the cell surface.

The present invention is explained in detail as follows. The present application claims the priority of Japanese Patent Application No. 2002-238559, which was filed on Aug. 19, 2002, and includes the contents as disclosed in the specification and/or drawings of the above patent application.

The present invention has been completed for the purpose of constructing carbohydrate library (sugar chain library, oligosaccharide library) containing various sugar structures and glycoforms. The present inventors have conducted studies focusing on the fact that cargo receptors recognize glycoproteins in the secretion pathway of glycoproteins in eukaryotic cells so as to be involved in the processing steps of carbohydrate moieties attached on glycoproteins. Hence, the present inventors have determined putative carbohydrate recognition domain of cargo receptors and have succeeded in modifying carbohydrate moieties of expressed glycoproteins by altering such domains.

1. Cargo Receptor

“Cargo receptor” is a general nomenclature for animal lectins playing important roles in quality control and sorting of glycoproteins. As cargo receptors, ERGIC-53 involved in transport of glycoproteins from the endoplasmic reticulum (ER) to the Golgi and VIP36 involved in quality control of glycoproteins in the Golgi (Hauri, H-P, et al., FEBS Letters 476 (2000) 32-37) are currently known. As shown in FIG. 1, after being synthesized in a form having carbohydrates added in the ER within eukaryotic cells, carbohydrate moieties (sugar moieties) of glycoproteins are subjected to processing in the Golgi. In secretory pathway, newly synthesized proteins are folded correctly in the ER and then transported between organelles by transport vesicles. During this process, proteins are glycosylated, sorted selected, and directed to move toward a site where each protein should function. Cargo receptors existing in the transport vesicles recognize carbohydrate moieties of glycoproteins and select proteins to be transported outside the cells.

During translation process, oligosaccharide is transfered to the Asf-Xaa-Ser/Thr peptide sequence (Xaa represents any amino acid other than proline) of proteins (Kornfeld R and Kornfelds, Ann Rev Biochem, 1985, 54: 631-664). The oligosaccharides added herein are oligosaccharides transferred from a lipid intermediate and contains 14 sugar units composed of mannose, glucose, and N-acetylglucosamine (GlcNAc). In the ER, the carbohydrate moiety of glycoproteins (e.g., a glucose residue located outermost part of the glycoprotein) is recognized by a lectin. Only the proteins correctly folded are subjected to removal of a glucose residue, and they are then transported to the Golgi for further modification (Teasdale R D, and Jackson M R, Annu Rev Cell Dev Biol, 1996, 12: 27-54).

Subsequently, in the ER-Golgi intermediate compartment (ERGIC, the zone between ER and the Golgi), ERGIC-53, one of the cargo receptors, functions in the transport of glycoproteins from the ER to the ERGIC. In the Golgi, further modifications of oligosaccharide are carried out by various glycosyltransferases and glycosidases, whereby finer oligosaccharide structures containing such as GlcNAc, galactose, fucose, and sialic acid are mainly formed. However, in this oligosaccharide processing, complete carbohydrate structures (glycoforms) are not always formed, and glycoproteins without functional activity are also generated. It is thought that VIP36 plays function in quality control of sugar chains attached on proteins and functions in the transport of glycoproteins from the trans-Golgi to the cis-Golgi or ERGIC (Fullekrug J, J Cell Sci, 1999, 112 (Pt 17): 2813-21). VIP36 prevents incomplete glycoproteins from being secreted by returning the incomplete glycoproteins to the initial stage of oligosaccharide processing in ERGIC or the cis-Golgi.

As described above, the term “cargo receptor” in the present invention is not specifically limited, as long as it recognizes sugar chains (carbohydrate moieties) attached on glycoproteins and is involved in the transport of glycoproteins required in vivo. In addition to recently discovered-ERGIC-53 and VIP36, cargo receptors that have similar functions and will be discovered in the future are also encompassed in the scope of the present invention.

The currently discovered 2 types of cargo receptors have already been isolated. Regarding ERGIC-53, human ERGIC-53 has been registered under GenBank accession number of X71661. Its nucleotide sequence is shown in SEQ ID NO: I and its amino acid sequence is shown in SEQ ID NO: 2. Furthermore, regarding VIP36, a human GP36b glycoprotein has been registered under GenBank accession number of U10362 (its nucleotide sequence is shown in SEQ ID NO: 3 and its amino acid sequence is shown in SEQ ID NO: 4) and canine (C. familiaris) VIP36 has been registered under GenBank accession number of X76392.

Cargo receptor is known to have a “carbohydrate recognition domain (also referred to as CRD)” at its central portion, which recognizes a glycoform (carbohydrate moiety) and binds to glycoproteins (Hauri, H-P. et al., Journal of Cell Science, 113:587-596, 2000). The present inventors have discovered that carbohydrate moieties of glycoproteins on the cell surface or secreted outside the cell can be modified by altering the carbohydrate recognition domain of a cargo receptor, so as to change the glycoform (carbohydrate moiety) to be recognized by the cargo receptor within the cells, resulting in a change in its quality control or sorting process for glycoproteins. The method for modifying a carbohydrate moiety of a glycoprotein and the method for preparing a cell expressing a glycoprotein with a modified carbohydrate moiety (with different glycoform) according to the present invention will be described as follows.

2. Alteration of Carbohydrate Recognition Domain of Cargo Receptor

(1) Carbohydrate Recognition Domain

Through comparison of homology with those of Leguminosae lectins, the present inventors have specified putative-carbohydrate binding domains, which are particularly important for determining carbohydrate (sugar chain) specificities of cargo receptors. The putative carbohydrate-binding domain corresponds to nucleotides from 454 to 480 of the nucleotide sequence of human ERGIC-53 cDNA (SEQ ID NO: 1; “a” of “atg” (translation initiation) is determined to be 1) and amino acids from 152 (Asp) to 160 (Lys) of ERGIC-53 amino acid sequence (SEQ ID NO: 2). The putative carbohydrate-binding domain of human VIP36 corresponds to nucleotides 484 to 510 of VIP36 cDNA (SEQ ID NO: 3) and amino acids 162 (Asp) to 170 (Thr) of VIP36 amino acid sequence (SEQ ID NO: 4). Carbohydrate-binding domains of non-human cargo receptors can also be assumed based on homology with human cargo receptors.

The present invention is characterized in that a carbohydrate recognition domain of a cargo receptor, particularly a carbohydrate-binding domain, is altered. In the present invention, the term “alteration of a carbohydrate recognition domain” means that when a cargo receptor is expressed as proteins, its carbohydrate recognition domain or its carbohydrate-binding domain differs from that of native one in terms of sequence and/or structure; or that a carbohydrate moiety (sugar chain) to be added differs from that of native protein to be expressed in cells. Hence, in addition to the above described specific sequences, alteration of a cargo receptor, whereby a carbohydrate moiety differing from that to be added before alteration, is also encompassed in the term “alteration of a carbohydrate recognition domain” according to the present invention.

In alteration of a carbohydrate recognition domain, any number of any amino acids can be changed and they are not particularly limited. However, when a carbohydrate recognition domain is altered by introducing mutations, among amino acid residues belonging to a carbohydrate recognition domain, aspartic acid (Asp) is important as an amino acid to be coordinated at Ca and Mn ions, and asparagine (Asn) is important because its side chain forms a cooperative hydrogen bond with sugars. Asparagine is also important as an amino acid residue to be coordinated and bound to Ca ions. This means that these amino acids may be greatly involved in keeping the structure of a carbohydrate-binding loop of cargo receptor and its binding to sugars. Among the above putative 9-amino acid-long loop determining carbohydrate-binding specificity, it is preferred not to introduce any mutations into amino acids 152 (Asp) or 156 (Asn) in the case of ERGIC-53, or into amino acids 162 (Asp) or 166 (Asn) in the case of VIP36, so as to conserve the amino acids.

(2) Introduction of Random Mutation Into Carbohydrate Recognition Domain

In the present invention, it is preferred to alter a carbohydrate recognition domain (or a carbohydrate-binding domain) of a cargo receptor by introducing mutations into its gene. A technique for introducing mutations into a partial region of a gene is known in the art. When correlation between mutations to be introduced and carbohydrate-binding specificities thus obtained is unknown, a carbohydrate recognition domain of a cargo receptor is randomly mutated, and then confirmed whether glycoforms (carbohydrate moieties) structures of glycoproteins is changed or not.

To introduce mutations randomly into a partial region of a gene, a random mutagenesis known in the art can be employed. Examples of such a technique are not limited and include a method using degenerate oligonucleotides, a linker scanning method, and a method based on PCR. Regarding these techniques, please see, for example, “Molecular Biology Experimental Protocol I” (particularly chapter 8) (Ausubel, F M et al., translated by Kaoru Saigo and Yumiko Sano, 1997, MARUZEN CO., LTD.) and “New Genetic Engineering Handbook” (particularly pages 216 to 226) (3rd revised edition, edited by Masami Muramatsu and Masashi Yamamoto, 1999, YODOSHA CO., LTD.). In the present invention, to achieve the purpose of preparing as many types of random oligonucleotides as possible in large quantities, a technique for introducing random mutations in combination with polymerase chain reaction (PCR method) using degenerate oligonucleotides as primers is preferred. Such a technique for altering a carbohydrate recognition domain of a cargo receptor by the PCR method using degenerate oligonucleotides will be described in detail as follows.

(2-1) Principle

The method using degenerate oligonucleotides is based on the phenomenon that degenerate oligonucleotides having random mutations can be synthesized by adding nucleotides other than normal nucleotides at the time of oligonucleotide synthesis. By utilization of this technique, degenerate oligonucleotides having various mutations introduced into regions corresponding to carbohydrate recognition domains of cargo receptors can be obtained. The method using degenerate oligonucleotides is advantageous in that the mutation rate can be controlled by increasing or decreasing the addition ratio of nucleotides (e.g., to obtain a mutation rate of 10% per nucleotide, 3 other nucleotides (3.33% each) are added to 90% of normal nucleotides).

Many kits are commercially available for preparing degenerate oligonucleotides. For example, the ExSite™ PCR-Based Site-Directed Mutagenesis Kit (Stratagene) or LA PCR™ in vitro Mutagenesis Kit (TaKaRa) can be used. When such a kit is utilized, various mutants having mutations such as a point mutation and deletion and/or insertion of several nucleotides can be conveniently prepared by changing primer design.

(2-2) Construction of Random Library

First, to obtain cargo receptor genes containing carbohydrate recognition domains with various mutations, random libraries of cargo receptor genes containing carbohydrate recognition domains wherein mutations have been randomly introduced are constructed using random primers comprising the above degenerate oligonucleotides.

As a random primer, a degenerate oligonulceotide hybridizing to a region around a carbohydrate recognition domain is used. A method for designing a random primer is known in the art, and a degenerate oligonucleotide can be easily determined by persons skilled in the art, as in the case of PCR reaction conditions and the like.

Random primers that can be utilized in the present invention are shown below, but they are not limited thereto:
Primers to amplify the latter half fragment on the 3′ terminal side containing a carbohydrate recognition domain of VIP36:
   VIPran3: 5′-CGT GCT CTA GAC NNK NNK NNK AAT NNK NNK NNK NNK GAG CGC GTG TTC CCG TA-3′, (SEQ ID NO: 7: in the sequence, N denotes A, T, G, or C and K denotes G or T)
   VIPran5: 5′-ATC GTC TTA AGC ACT CAG TAG AAG CGC TTG-3′ (SEQ ID NO: 8)
Primers to amplify the latter half fragment on the 3′ terminal side containing a carbohydrate recognition domain of ERGIC-53 (in the sequence, the underlined site denotes a restriction enzyme site):

ERGIC-BF: 5′-CGTATCTAGATXXKXXKXXKAATXXKXXKXXKXXKA ATAATCCTGCTATAGTAATTAT-3′ (SEQ ID NO: 14: in the sequence, K denotes G or T, and X denotes any one of A, T, G, or C)

(SEQ ID NO: 15)

ERGIC-BR: 5′-CGTACTTAAGTGGTAGTCAAAAGAATTTTTTG3′

A method for synthesizing primers designed as described above is known in the art. For example, a general oligonucleotide synthesis method such as a phosphoamidite method can be employed.

Next, through the use of the above-designed primers, amplification reaction is carried out using a cargo receptor cDNA or a cDNA library or mRNA as a template. Examples of amplification reaction are not limited and include polymerase chain reaction (PCR) and a LAMP method (Loop-mediated Isothermal Amplification). Extraction of mRNA and construction of a cDNA library can be carried out according to conventional methods.

Using the thus obtained mRNA as a template, single-stranded DNA can be synthesized using random primers and reverse transcriptase. Double stranded DNA is then synthesized from the single-stranded DNA. In the case of the PCR method, double-stranded DNA can be obtained. Subsequently, the thus obtained double-stranded DNA is incorporated into an appropriate cloning vector so as to construct a recombinant vector. Then, the recombinant vector can be transformed into Escherichia coli or the like, and then transformants are selected using tetracycline resistance, ampicillin resistance, or the like as an indicator, so that random libraries can be obtained.

Next, a portion containing a target cargo receptor gene can be cloned from the obtained clones. For DNA cloning, for example, a TA cloning method can be employed. The TA cloning method can be conducted using a commercially available kit such as a TA cloning kit (Invitrogen Corporation).

For the isolated DNA clones obtained in the above screening, the DNA nucleotide sequences are determined using amplification products as templates.

Nucleotide sequences can be determined by a known techniques such as Maxam and Gilbert's chemical modification method or the dideoxynucleotide chain termination method using an M13 phage. In general, sequencing is carried out using an automated system for determining nucleotide sequences (e.g., DNA Sequencer LONG READER 4200 produced by LI-COR, INC.).

In addition to the above-described methods, based on the nucleotide sequence of a cargo receptor gene, through the use of technology for artificially deleting, substituting, or inserting one to several nucleotides into the nucleotide sequence, such as the site-directed mutagenesis method, mutants having carbohydrate recognition domains with different sequences while maintaining the functions of a cargo receptor can be prepared. For example, for site-directed mutagenesis whereby one to several nucleotides are substituted, mutants can be obtained and utilized according to the technology as described in, for example, Proc. Natl. Acad. Sci. USA 81 (81984) 5662-5666, WO85/00817 (PCT Pamphlet), Nature 316 (1985) 601-605, Gene 34 (1985) 315-323, Nucleic Acids Res. 13 (1985) 4431-4442, Proc. Natl. Acad. Sci. USA 79 (1982) 6409-6413, or Science 224 (1984) 1431-1433. Moreover, these mutants can be prepared through the use of a commercially available kit (Mutan-G and Mutan-K (Takara)). Furthermore, error-prone polymerase chain reaction (error-prone PCR) is also known as a method for preparing mutants. In this method, mutation of one to several nucleotides can be introduced by selecting conditions with a low degree of strictness (fidelity) for amplification (Cadwell, R. C. and Joyce, G. F. PCR Methods and Applications 2 (1992) 28-33; Malboeuf, C. M. et al. Biotechniques 30 (2001) 1074-8; Moore, G. L. and Maranas C. D. J. Theor. Biol. 7; 205 (2000) 483-503).

In the case of random mutations, for example, among nucleotides corresponding to a carbohydrate recognition domain of VIP36, 14 nucleotides are each of A, T, G, or C, and 7 nucleotides are each of G or T, so that at least 4¹⁴×2⁷ (that is, 3.4×10¹⁰) combinations of sequences into which mutations have been randomly introduced are possible. Furthermore, for example, among 9 amino acids corresponding to a carbohydrate recognition domain of ERGIC-53, mutations can be introduced into 7 amino acids. Hence, 14 nucleotides are each of A, T, G. or C and 7 nucleotides are each of G or T, so that there are at least 4¹⁴×2⁷ (that is, 3.4×10¹⁰) combinations of sequences into which mutations have been randomly introduced, as in the case of VIP36. Moreover, by the introduction of mutations such as those involving deletion or insertion, the resulting random libraries will contain a greater number of various sequences.

(2-3) Random Vector

A vector for cell transfection is constructed using the above-obtained random libraries of cargo receptor genes containing carbohydrate recognition domains having various mutations.

A random vector to be transfected into cells can be obtained by ligating DNA of the above random library into an appropriate vector. A transfected cell can be obtained by introducing the above random vector into a host so that a target cargo receptor gene can be expressed.

As a vector, a vector known in the art as a vector for transfection can be used. Examples of such a vector include plasmid DNA, phage DNA, animal virus vectors e.g., retroviruses or vaccinia viruses, insect virus vectors e.g., baculoviruses, bacterial artificial chromosome (BAC), and yeast artificial chromosome (YAC).

To insert a cargo receptor gene of the random libraries into a vector, for example, a method that involves cleaving a purified DNA with an appropriate restriction enzyme and inserting the DNA into a restriction enzyme site or a multi-cloning site of an appropriate vector DNA so as to ligate the DNA to the vector can be employed.

It is necessary to incorporate a cargo receptor gene into a vector so that the functions of the gene can be exerted. Hence, in addition to a promoter and a cargo receptor gene, a cis-element such as an enhancer, a splicing signal, a polyA addition signal, a selection marker, a ribosome-binding sequence (SD sequence), or the like can be ligated to a recombinant vector, if desired. In addition, examples of a selection marker include a dihydrofolate reductase gene, an ampicillin resistance gene, and neomycin resistance gene.

To ligate a DNA fragment to a vector fragment, a known DNA ligase can be used. Next, a DNA fragment and a vector fragment are annealed and then ligated, so that a random vector is constructed.

(2-4) Transfection Into Cell

Hosts used for transfection are not specifically limited, as long as they are eukaryotic cells. Examples of such a host include yeast, animal cells (e.g., COS cells, CHO cells, or MDCK cells), and insect cells. When the present invention is applied to production of proteins derived from an animal such as a human, it is particularly preferable to use animal cells.

When yeast is used as a host cell, for example, Saccharonmyces cerevisiae, Schizosaccharomyces pombe, or the like is used. In this case, a promoter is not particularly limited, as long as it can direct a gene expression in yeast. A method for introducing a recombinant vector into yeast is not particularly limited, as long as it is a method for introducing DNA into yeast. Examples of such a method include an electroporation, a spheroplast method, and a lithium acetate method.

When an animal cell is used as a host, simian COS-7 cells, simian Vero cells, Chinese hamster ovary cells (CHO cells), mouse L cells, rat GH3, human FL cells, Mardin Darby canine kidney cells (MDCK cells), or the like are used. As a promoter, SRα promoter, SV40 promoter, LTR promoter, CMV promoter, or the like is used. In addition, for example, a promoter of human cytomegalovirus early gene may be used. Examples of a method for introducing a recombinant vector into an animal cell include an electroporation method, a calcium phosphate method, and a lipofection. A method for introducing a vector into an animal cell can be conveniently carried out using a commercially available kit, such as Effectene® transfection reagent (QIAGEN) or LipofectAMINE reagent (Invitrogen) according to the manufacturer's protocols.

When an insect cell is used as a host, Sf9 cells or the like can be used. Examples of a method for introducing a recombinant vector into an insect cell include a calcium phosphate method, a lipofection, and an electroporation.

Transfected cells can be selected utilizing the properties of a marker gene that is a constituent of a gene to be introduced. For example, when a neomycin resistance gene is used, cells exhibiting resistance against the G418 drug are selected.

Whether or not a target cargo receptor gene is incorporated into cells can be confirmed by the PCR method, the Southern hybridization method, or the like. For example, DNA or mRNA is prepared from transfected cells, primers specific to an introduced DNA are designed, and then PCR is performed. Subsequently, amplification products are subjected to agarose gel electrophoresis, polyacrylamide gel electrophoresis, capillary electrophoresis, or the like stained using ethidium bromide, SYBR Green fluid, or the like, and then detected as a single band, so that the introduced DNA can be confirmed. Furthermore, PCR is performed using primers previously labeled with fluorescent dye or the like, so that amplification products can also be detected. Furthermore, a method that involves causing amplification products to bind to a solid phase such as a micro plate and confirming the amplification products by fluorescence reaction, enzyme reaction, or the like can be employed.

(3) Separation

After alteration of a carbohydrate recognition domain of a cargo receptor as described above, cells expressing a specific glycoprotein with a modified carbohydrate moiety (different glycoform) can be separated based on its carbohydrate moiety (glycoform).

A method for separating transfected cells based on the specific carbohydrate moiety is not particularly limited, as long as it is based on techniques for identifying a carbohydrate moiety (glycoform) known in the art. In the present invention, because of its convenience, cells are preferably separated by identifying a carbohydrate moiety of a glycoprotein expressed on the cell surface of each transfected cell utilizing a plural number of types of plant lectins having carbohydrate-binding specificities.

Examples of plant lectins include Agarictis bisporus (ABA) lectin, Jack bean (Canavalia ensiformis) (ConA) lectin, Datura stramonium (DSA) lectin, Lens culinaris (LCA) lectin, Lotus Itetragonolous (Lotus) lectin, Maackia amurensis (MAM) lectin, Phaseolus vulgaris lectin having homotetramer E-subunits (PHA-E₄), Phaseolus vulgaris lectin having homotetramer L-subunits (PHA-L₄), Caster bean (RCA120) lectin, and wheat germ (WGA) lectin.

Detailed studies have been conducted concerning plant lectins. For example, as shown in the following Table 1, the sugar specificities have been precisely analyzed.

TABLE 1

| Lectin | Sugar (carbohydrate) specificity | Carbohydrate type |
|---|---|---|
| ABA | D-Gal | O-linkage |
| ConA | α-D-Man, α-D-Glc | N-linkage |
| DSA | β-D-GlcNAc, (β1-4GlcNAc)ₙ | |
| LCA | α-D-Glc, α-D-Glc, α-D-Man | N-linkage |
| Lotus | α-L-Fuc | |
| MAM | SAα2-3Gal | N- or O-linkage |
| PHA-E₄ | D-GalNAc | N-linkage |
| PHA-L₄ | D-GalNAc | N-linkage |
| RCA120 | β-D-Gal | N- or O-linkage |
| WGA | D-GlcNAc (bisecting) | N- or O-linkage |

As a plant lectin, for example, as a biotinylated lectin, Biotin-Lectin set I and II (HONEN Corporation (J:OIL MILLS, Inc.)) are commercially available.

Regarding studies on plant lectins and oligosaccharides or carbohydrates, on the homepage of HONEN Corporation, with reference to protocols for carbohydrate analysis and lectin-related information, plant lectins other than those listed in Table 1, sugar specificities thereof, techniques for purifying glycoproteins are published (see http://www.honen.co.jp/finechem/; http://www.j-oil.com/finechem/).

The type of a modified carbohydrate moiety can be determined utilizing sugar specificities of the above plant lectins. A method for detecting cells having carbohydrate moieties bound to specific plant lectins is not particularly limited, as long as it is a technique known in the art. For example, such cells can be detected by a technique for detecting a labeled-plant lectin, or the like.

Preferably, to separate cells having carbohydrate moieties bound to labeled plant lectins, a technique (Magnetic Cell Sorting; MACS method) that involves performing secondary labeling using magnetic beads and separating cells utilizing magnetism, or a technique that involves sorting cells by flow cytometry based on the information of labeling can be employed (Fluorescent Cell Sorting; FACS method). When flow cytometry is utilized, cells can be sorted by analyzing labels of individual cells, so that cells having specific carbohydrate moieties can be precisely separated one by one. In the case of MACS, procedures thereof are convenient and can be performed in a short time, resulting in less damage to cells and low risk of bacterial contamination, or the like. Furthermore, regarding the above techniques for separating cells, 1 type of technique may be conducted once or several times, or 2 or more types of techniques may be combined and respectively conducted once or several times.

By subculturing of the above-separated cells, cells having specific carbohydrate moieties can be concentrated (enriched).

3. Confirmation of Altered Domain and Modified Carbohydrate Moiety

As described above, correlation between altered domains and modified carbohydrate moieties can be elucidated by randomly introducing mutations into carbohydrate recognition domains of cargo receptors and then examining the carbohydrate structures (glycoforms) of the obtained glycoproteins.

Therefore, when specific alteration is carried out to obtain a specific modified carbohydrate moiety, mutations are introduced site-specifically into a carbohydrate recognition domain of a cargo receptor. As such a technique, a known technique such as a Kunkel method or a Gapped duplex method, or a related method can be employed. Mutations can be conveniently introduced using, for example, a kit for mutation introduction utilizing the site-directed mutagenesis method (e.g., Mutan-K (TAKARA BIO INC.) and Muran-G (TAKARA BIO INC.)).

As described above, specific mutations are introduced into a carbohydrate recognition domain of a cargo receptor, or mutations are randomly introduced into a carbohydrate recognition domain of a cargo receptor, and then cells expressing proteins having the desired type of carbohydrate moiety are selected. Thus, causing proteins having desired modified carbohydrate moieties to be expressed on the cells becomes possible, and thus the modified carbohydrate moieties (or modified oligosaccharide) can be conveniently obtained in large quantities.

4. Preparation of Cell Expressing Glycoprotein Having Modified Carbohydrate Moiety

As described above, when correlation between an altered domain and a modified carbohydrate moiety is determined, cells expressing glycoproteins having the specific modified carbohydrate moiety can be prepared. Alternatively, such cells can also be obtained by selecting cells expressing glycoproteins having the specific modified carbohydrate moiety from among cells wherein mutations have been randomly introduced into a carbohydrate recognition domain of a cargo receptor. In the present invention, the term “modified carbohydrate moiety” or “modified carbohydrate structure” indicates that the sequence and/or the structure of such a carbohydrate moiety differs from that of an unaltered cargo receptor. Furthermore, in the present invention, the term “glycoprotein” or “protein with or having a carbohydrate moiety” indicates a substance composed of sugars (carbohydrates) and proteins that are covalently bound to each other.

Proteins to which a specific modified carbohydrate moiety is bound is expressed in the above-prepared cell by conventional genetic engineering techniques so that the carbohydrate moiety can be bound to the protein. Examples of such proteins include, but are not limited to, proteins to be used as pharmaceutical products such as erythropoietins (EPO), granulocytic colony-stimulating factors (G-CSF), interleukins, and antibodies. Regarding EPO, it has already been reported that modification of the carbohydrate moiety leads to the prolonged half-life of the glycoprotein in blood. Regarding an antibody, it has also been reported that modification of the carbohydrate moiety leads to enhanced activity of the protein. Hence, oligosaccharide (carbohydrate) processing may be able to confer effects equivalent to or better than the properties (effects) of proteins or other pharmaceutically useful properties (effects) to proteins. Moreover, proteins to which a specific modified carbohydrate moiety (oligosaccharide) is bound is not limited to proteins for pharmaceutical use. For example, proteins subjected to studies on the in vivo effects of carbohydrate moieties on the proteins, T cell receptors, NK cell receptors, chemokine receptors, and MHC class I and II molecules involved in intercellular recognition, adhesion molecules such as NCAM (neural cell adhesion molecule), cadherin, integrin, LFA-1 (lymphocyte function-related antigen-1), ICAM-1 (intercellular adhesion molecule-1), and GlyCAM-1, activin, Notch, Delta, and Serrate involved in development, extrcellsular matrices such as mucin, and collagen are also encompassed in the present invention.

To express a desired protein in the above-prepared cell, for example, as described in sections (2-3) and (2-4) of “2. Alteration of carbohydrate recognition domain of cargo receptor,” a gene encoding the protein is inserted into a recombinant vector, and then the vector is transformed into or transfected into cells. At this time, to express the protein as a membrane-bound protein, in addition to a gene encoding the protein, a fusion gene is constructed to encode a secretory signal sequence for transporting the protein to the cell surface and a sequence of a transmembrane domain of cells surface-localized protein or a GPI anchor sequence on the same gene. The fusion gene is then expressed in cells. Furthermore, to express the protein as a secreted protein, a fusion gene encoding the gene sequence that encodes the protein and a secretory signal sequence on the same gene is constructed, and then the fusion gene is expressed in cells.

A secretory signal sequence (also referred to as a secretory signal or a secretory signal peptide) is generally bound to the N-terminus of proteins to be secreted outside the cell. In general, the sequence is removed when proteins to be secreted outside the cell is secreted from the inside to the outside of the cell via the cell membrane. In the present invention, any secretory signal sequences that can transfer and express a desired protein outside the cell can be used.

Cell surface-localized proteins are those that are fixed on the cell surface layers of host cells and that exist on the cell surface. The cell surface means any of the inside of the outermost membrane (e.g., cell wall and cell membrane) of a host cell, the interface between the outermost layer and the outside of the cell membrane, and regions protruding via linkers or anchors from the outermost layer of cells. Cell surface-localized proteins are not particularly limited, as long as they enable fixation of desired proteins on the cell surface layers.

When a desired protein is expressed using the cell expressing the glycoproteins with a modified carbohydrate moiety (different glycoform) according to the present invention, the modified carbohydrate moiety is added to the protein. Hence, according to the present invention, a specific carbohydrate moiety can be bound to a desired protein.

5. Glycoproteins With a Modified Carbohydrate Moiety

In the present invention, through the use of cells prepared as described in the above sections “2. Alteration of carbohydrate recognition domain of cargo receptor” or “4. Preparation of cell expressing glycoprotein having modified carbohydrate moiety,” a glycoprotein with a modified carbohydrate moiety (with different glycoform) can be produced.

In the present invention, a glycoprotein with a modified carbohydrate moiety can be obtained by culturing the above-prepared cells and collecting the protein from the culture. The term “culture” means any of cultured cells or disrupted cells. A method for culturing transfected cells in media is conducted according to a method generally employed for culturing host cells.

As a medium for culturing a transformant obtained using a microorganism such as yeast as a host, either a natural or a synthetic medium may be used, as long as it contains carbon sources, nitrogen sources, inorganic salts, and the like, assimilable by microorganisms, and enables efficient culture of transformants. Here, carbon sources, nitrogen sources, inorganic substances, and the like to be added to media are known in the art.

Culturing is generally carried out under aerobic conditions such as shake culture or aeration and agitation culture at approximately 28° C. to 40° C. for approximately 18 hours to 10 days. During culturing, a roughly neutral pH is maintained, such as pH 7.4. pH is adjusted using inorganic or organic acid, alkaline solution, or the like. During culturing, antibiotics such as ampicillin or tetracycline may be added to media, if necessary.

When a microorganism transformed with an expression vector having an inducible promoter used therein as a promoter is cultured, an inducer may be added to media, if necessary. For example, when a microorganism transformed with an expression vector having a Lac promoter used therein is cultured, isopropyl-β-D-thiogalactoside (JPTG) or the like may be added to media. When a microorganism transformed with an expression vector having a trp promoter used therein is cultured, indoleacetic acid (IAA) or the like may be added to media.

As media for culturing transfected cells obtained using animal cells as host cells, for example, generally-employed RPMI1640 media, DMEM media, or HAM F10 media supplemented with 5% to 20% fetal bovine serum (FBS) or various commercially available serum-free media can be used. Culturing is generally carried out in the presence of 5% CO₂ at 37° C. for approximately 18 hours to 10 days. During culturing, antibiotics such as kanamycin or penicillin may be added to media, if necessary.

After culturing, a target glycoprotein with a modified carbohydrate moiety (with different glycoform) can be obtained using a general means for purifying proteins. When the protein is a membrane-bound protein, since such proteins is produced on cell surface the proteins are extracted by disrupting and/or solubilizing the cells. Furthermore, when the protein is a secreted protein, the proteins are collected from the culture supernatant. Subsequently, the target proteins can be isolated and purified from the above culture using one of or an appropriate combination of general biochemical methods that are employed for protein isolation and purification, such as ammonium sulfate precipitation, gel chromatography, ion exchange chromatography, and affinity chromatography.

Whether or not a target protein is obtained can be confirmed by SDS-polyacrylamide gel electrophoresis or the like.

Methods for producing a glycoprotein with a modified carbohydrate moiety utilizing cells are as described in detail above. As is known by persons skilled in the art, it is also possible to produce a glycoprotein with a modified carbohydrate moiety by modifying carbohydrate moiety in cell-free system. A kit for producing proteins utilizing cell-free system (e.g., an in vitro translation system) is commercially available. The present invention can be achieved utilizing such a kit. Briefly, through the utilization of an in vitro transcription/translation system such as TnT® Coupled Reticulocyte Lysate Systems (rabbit reticulocyte) or TnT® Coupled Wheat Germ Extract Systems (wheat germ) marketed by Promega Corporation, for example, a cargo receptor having an altered carbohydrate recognition domain and a desired protein are expressed, so that the modified carbohydrate moiety is bound to the desired protein. For details of the in vitro translation systems of Promega Corporation, please see the homepage thereof (http://www.promega. com/guides/ive_guide/default.htm).

As described above, the present invention is not limited to the production of a glycoprotein with a modified carbohydrate moiety in a specific cell system.

6. Production of Oligosaccharide

Once a glycoprotein with a modified carbohydrate moiety is obtained as described above, an oligosaccharide can be cleaved from the protein, so that the modified oligosaccharide can be obtained. Cleavage of such an oligosaccharide can be carried out by techniques known in the art, such as a hydrazinolysis method or degradation by enzymes. Regarding the hydrazinolysis method, a Hydraclub hydrazinolysis reagent C (HONEN Corporation) is marketed, by which oligosaccharides can be cleaved according to the instructions. Regarding degradation by enzymes, for example, N-glycopeptidase or O-glycanase is allowed to react with glycoproteins, so that the oligosaccharide can be cleaved.

According to the present invention, a desired carbohydrate moiety or oligosaccharide can be produced conveniently and in large quantities. Carbohydrates and oligosaccharides are required in many fields including biological and chemical fields; however, the production thereof has required much time and economic cost. The present invention is very useful in every field that requires such carbohydrates or oligosaccharides.

7. Carbohydrate Library (Sugar Shain Library)

As shown in (2-2) above, a random library including sequences obtained by randomly introducing mutations into a cargo receptor is very large. Hence, by the transfection of cells using the random library of the cargo receptor, a library of cells expressing glycoproteins with different glycoforms (modified carbohydrate moieties) on the cell surfaces can be constructed. In addition, the library may be composed of modified oligosaccharides or glycoproteins with different glycoforms prepared according to the present invention.

Since it is thought that the structures of carbohydrate moieties or oligosaccharides are involved in vivo functions, the carbohydrate library is useful for carbohydrate studies. For example, a carbohydrate moiety or oligosaccharide having important functions can be searched for from the carbohydrate library, which include various carbohydrate moieties or oligosaccharides.

To select (screen for) a carbohydrate moiety or oligosaccharide having a specific structure or a specific function from the carbohydrate library, for example, a technique for selecting (screening for) a carbohydrate moiety or oligosaccharide based on its binding to a plant lectin, an antibody, and a receptor or a technique for selecting in vivo radio-labeled cells based on their accumulation in organs (targeting) can be employed.

Furthermore, the carbohydrate library enables the use of cells expressing various glycoproteins, or oligosaccharides or glycoproteins immobilized on chips. Such chips are useful for extensively detecting (screening for) a carbohydrate moiety or oligosaccharide having a specific structure or a specific function. In contrast, oligosaccharides or glycoproteins having specific carbohydrate structures or cells can be previously selected from the carbohydrate library, immobilized respectively on various fluorescence beads (Luminex Corporation) or chips, and then used. Such fluorescence beads or chips are useful for extensively detecting (screening for) substances (e.g., proteins) or cells recognizing specific carbohydrate structures. In the present invention, the term “screening” means a step of selecting candidates such as substances having specific carbohydrate structures or substances specifically interacting with specific carbohydrate structures, and indicates the sifting out of numerous carbohydrate structures in a carbohydrate library so as to narrow down candidate substances, or the narrowing down of candidate substances interacting with cells expressing glycoproteins having specific structures in a carbohydrate library. Specifically, the screening method of the present invention comprises, for example, the following steps of:
(a) bringing a cell expressing a glycoprotein with a modified carbohydrate moiety or a modified oligosaccharide or a glycoprotein with a modified carbohydrate moiety into contact with a test substance; and
(b) examining interaction of the glycoprotein or the carbohydrate moiety (oligosaccharide) with the test substance.

In step (a), the method for contacting is not particularly limited. For example, a cell expressing a glycoprotein with a modified carbohydrate moiety or a modified oligosaccharide or a glycoprotein with a modified carbohydrate moiety can be brought into contact with a test substance by mixing them. Furthermore, in the step (b), the method for examining interaction is not particularly limited, either. Interaction can be examined utilizing various methods known by persons skilled in the art.

The above screening method may also include a step of growing cells expressing a glycoprotein with a modified carbohydrate moiety and/or a step of screening for a cell expressing a desired glycoprotein or a carbohydrate moiety on the cell surface. For example, cells expressing a glycoprotein with a modified carbohydrate moiety included in a carbohydrate library can be grown according to a general cell growth method, so as to be able to increase the amount of the modified carbohydrate moiety. Furthermore, since a glycoprotein with a carbohydrate moiety is expressed on cells in the present invention, a specific carbohydrate moiety or oligosaccharide can also be studied utilizing techniques such as flow cytometry.

Proteins recognizing specific carbohydrate moieties are often involved in various diseases, including viral infections, bacterial infections, and the like. Thus it is desired to screen for and discover such proteins. Moreover, it is known that infection is established by the binding of specific oligosaccharides on viruses or bacteria having specific carbohydrate moieties to cells. It is also known that contact of specific carbohydrate moieties with antibodies or cells regulates the functions of such antibodies or cells. It has been desired to examine such regulatory mechanisms. Hence, it is expected that the in vivo regulatory mechanisms of proteins recognizing specific carbohydrate moieties or of specific carbohydrate moieties will be discovered according to the present invention, and that then target molecules (sugars) of drugs for diseases or diagnostics will become clear, thereby enabling efficient design of drugs with no side effects and higher level diagnosis of diseases.

### BRIEF DESCRIPTION OF DRAWINGS

FIG. 1 shows cargo receptors (ERGIC-53 and VIP36) involved in carbohydrate processing of glycoproteins and the quality control of carbohydrates (sugars) and the outline of secretory pathway.

FIG. 2 shows the outline of VIP36 library construction.

FIG. 3 shows the result of introducing random mutations into VIP36. “A” shows the nucleotide sequences of the putative carbohydrate-binding domains of VIP36 and “B” shows the amino acid sequences thereof.

FIG. 4 shows photographs of overexpression of altered VIP36 in MDCK cells.

FIG. 5 shows the binding of plant lectins to lectin-positive (red) and lectin-negative (black) MDCK cells. The upper row shows untransfected cells, the middle row shows mutated VIP36 transfected cells, and the lower row shows untransfected control MDCK cells.

FIG. 6 shows the binding of PHA-E₄ and WGA to lectin-positive (red) and lectin-negative (black) MDCK cells. The uppermost row shows wild-type cells, the second row from the top shows mutated VIP36 transfected cells resulting from the 1^{st} separation, the second row from the bottom shows mutated VIP36 transfected cells resulting from the 2^{nd} separation, and the lowermost row shows the mutated VIP36 transfected cells resulting from the 3^{rd} separation.

FIG. 7 shows the effect of trypsinization on the binding of lectins. PBS-EDTA treatment is indicated with red, and trypsin-EDTA treatment is indicated with blue.

FIG. 8 is an outline showing the design of random primers for introducing random mutations into the carbohydrate-binding domain of ERGIC-53.

FIG. 9A shows a photograph showing samples prepared using an anti-FLAG antibody as an primary antibody and a goat anti-mouse IgG₁-FITC as a secondary antibody and then observed by a fluorescence microscope. As a negative control, wild-type MDCK cells were observed.

FIG. 9B shows a photograph showing samples prepared using an anti-FLAG antibody as a primary antibody and goat anti-mouse IgG₁-FITC as a secondary antibody and then observed by a fluorescence microscope. As a positive control, VIP36-FLAG clone8 (clones whose constant expression has been confirmed) was observed.

FIG. 9C shows photographs showing samples prepared using an anti-FLAG antibody as a primary antibody and goat anti-mouse IgG₁-FITC as a secondary antibody and then observed by a fluorescence microscope. ERGIC random libraries were transfected into MDCK cells, and then selection was carried out for 10 days with 1.5 mg/ml G418, and then the cells were observed.

FIG. 10 shows histograms showing the result of analyzing the cells following MACS screening using various biotinylated lectins as primary antibodies and FITC-labeled streptavidin as a secondary antibody. A black line indicates control MDCK cells, a red line indicates a (−) fraction following MACS, and a green line indicates a (+) fraction.

FIG. 11 shows a histogram showing the result of analyzing by FACS MDCK cells fractionated by MACS using PNA lectins. The binding strength of PNA (−), that of PNA (+), and that of PNA2 (+) to PNA were compared.

FIG. 12 shows photographs showing the results of western blotting carried out for PNA (−), PNA (+), and PNA2 (+) using 5 types of biotinylated lectins as primary antibodies and streptavidin alkaline phosphatase as a secondary antibody.

FIG. 13 shows the result of analyzing the carbohydrate-binding specificity of each cell fraction when FACS was carried out for a control, PNA (−), PNA (+), and PNA2 (+) using MAM or PNA as a primary antibody and streptavidin FITC as a secondary antibody.

FIG. 14 shows the outline of a technique for separating cells having specific carbohydrate moieties by flow cytometry or a magnetic cell sorting (MACS) using labeled lectins.

FIG. 15 shows the intensity of fluorescence for labeled PNA lectins in a process where cells having carbohydrate moieties specifically binding to PNA lectins were separated by flow cytometry (FACS) and then enriched.

FIG. 16 shows the expression of carbohydrate moieties to be recognized by altered VIP36 and PNA lectins in clone 12 and control CHO cells.

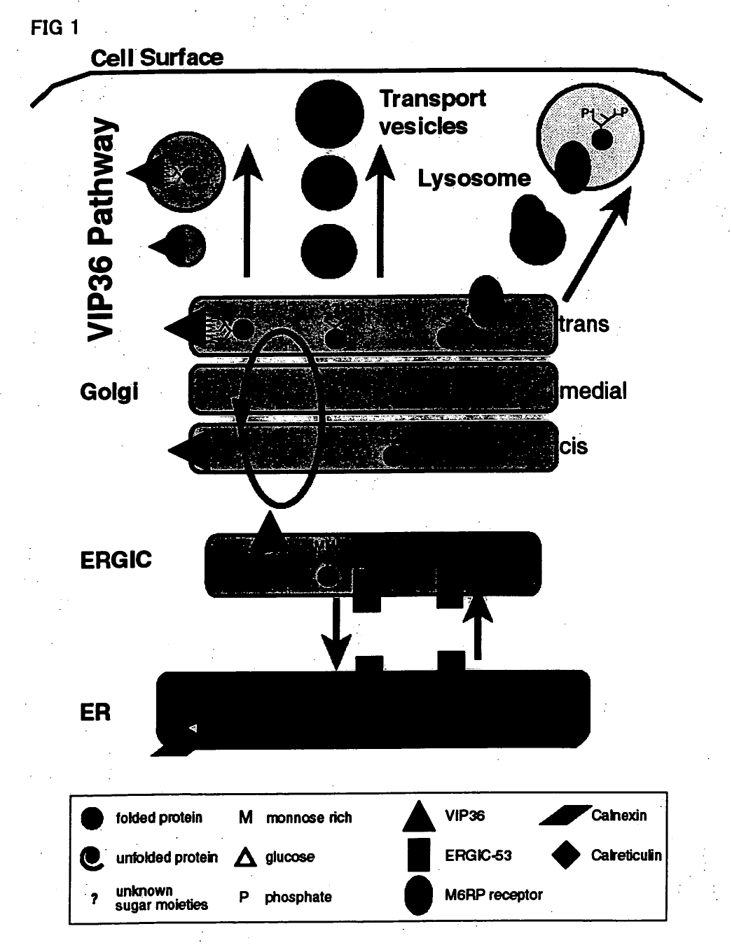

### BEST MODE OF CARRYING OUT THE INVENTION

The present invention will be hereafter described in detail by referring to examples, but the present invention is not limited by these examples.

### EXAMPLE 1

### Construction of VIP36 Random Library

(1) Plasmid pRc/CMV2-flag-VIPh-AflII

In this example, in order to introduce random mutations into a portion of cDNA encoding a carbohydrate-binding domain of VIP36, the following primers were designed with randomised oligonucleotides, overlapping cDNA encoding the carbohydrate-binding domain so that when polymerase chain reaction (PCR) was performed, the amplified cDNA fragments (named VIPt, nucleotide from 481 to 1071 of total size of 1407 nucleotides) contain randomly mutated carbohydrate-binding domain.

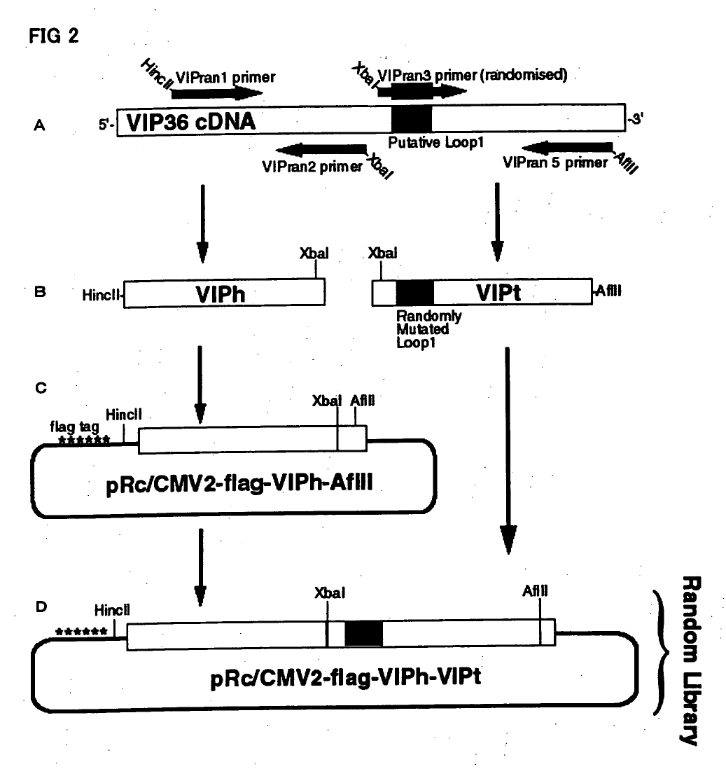

The following four primers were used for constructing VIP36 random library (FIG. 2A): VIPran1: 5′-GCA TGT CGA CAT AAC TGA CGG CAA CAG TG-3′ (SEQ ID NO: 5; with restriction site HincII: GTC GAC included at 5′ end of nucleotides), VIPran2: 5′-GAG CTC TAG AAA GAT GGC TAA GCC GTG GAA-3′ (SEQ ID NO: 6; with XbaI site: TCT AGA at 5′ end), VIPran3: 5′-CGT GCT CTA GAC NNK NNK NNK AAT NNK NNK NNK NNK GAG CGC GTG TTC CCG TA-3′, SEQ ID NO: 7; where N is mixture of A/T/G/C, and K mixture of G/T, with XbaI site at 5′ end), and VIPran5: 5′-ATC GTC TTA AGC ACT CAG TAG AAG CGC TTG-3′ (SEQ ID NO: 8; with BfrI site: CTT AAG at 5′ end).

VIPran1 and VIPran2 primers were used in PCR to construct 5′ half of VIP36 gene (VIPh fragment, 133-480 nucleotides of the entire length of 1407 nucleotides). Primers were first phosphorylated in the mixture of: 10 μl of VIPran1 (2 μg/μl), 10 μl of VIPran2 (2 μg/μl), 2 μl of 10× Kination buffer A (New England Biolab), 1.5 μl of 10 mM ATP, 6 μl of water, 1.5 μl of T4 Kinase. The solution was kept at 37° C. for 2 hours. PCR was performed with reaction solution consisting of: 1.5 μl of each phosphorylated primer, 5 μl of 10×KOD plus buffer (TOYOBO), 2 μl of MgSO₄ (TOYOBO), 2 μl of 2 mM dNTP mix, 2 μl of pBluescript-VIP36 (as template, 10 ng/μl), 3.5 μl of water and 1 μl of KOD plus (TOYOBO). PCR programme was run in either PCRexpress (Hybaid) or GeneAmp PCR system 2400 (Perkin Elmer), beginning with 94° C. for 2 min, followed by 30 cycles of 20 s at 94° C., 30 s at 52° C. and 1 min at 68° C., then kept at 68° C. for 5 min. Amplified VIPh fragment was harvested by 1% agarose gel electrophoresis, subjected to gel extraction (QIAquick Gel Extraction Kit, QIAGEN), and stored in TE buffer (FIG. 2B).

VIPh fragment was ligated with pBluescript (pBluescript SK(+) (Stratagene)) at 15° C. overnight after SmaI digestion followed by alkaline phphatase treatment. Thus, prepared mixture was transformed into CaCl₂ treated E. coli strain JM109 by heatshock method, and cultured on a LB-Amp plate with 50 μl of 5-bromo-4-chloro-3-indolyl-beta-galactoside (X-Gal) and 4 μl of isopropyl-1-thio-beta-D-galactoside (IPTG) for colour selection. Plate was incubated for over night. White-coloured colonies were selected and cultured overnight in LB medium in the presence of 100 μg/ml of ampicillin (LB-Amp medium). Plasmid DNAs were extracted from overnight-cultured medium using Plasmid Mini Kit (QIAGEN) and stored in TE buffer. pBS-VIPh vector was then digested with HincII and NotI in pBluescript containing VIPh fragment. Digested VIPh fragments (VIPh′, with a few pBluescript nucleotides at 3′ end, cut with HincII and NotI at each ends) were extracted using QIAquick Gel Extraction Kit and stored in TE buffer. VIPh′ fragments were ligated at 15° C. overnight with pRc/CMV2-frag which had been digested with HpaI and NotI (HpaI site and NotI site present in flag tag and pRc/CMV2, respectively). pRc/CMV2-flag (5641 bp) was a altered pRc/CMV2 (Catlog No. V750-20, Invitrogen) which flag sequence 5′-GAC TAC AAA GAC GAT GAC GAC AAG-3′ (SEQ ID NO: 9) was inserted into between HincII (base pairs 1981 of sequence size 5641 bp) and HpaI restriction sites of pRc/CMV2. Ligated mixture was transformed into E. coli and cultured on a LB-Amp agar plate at 37° C. overnight. Four colonies were picked up and cultured in LB-Amp medium respectively at 37° C. for overnight. pRc/CMV2-flag-VIPh was harvested by Plasmid Mini Kit, and then sequenced.

For sequencing pRc/CMV2-flag-VIPh, dideoxy method with 8.5 μl of total solution consisting of: 0.75 μl of pRc/CMV2F (forward dye primer), 0.75 μl of pRc/CMV2R (reverse dye primer), 0.5 μl of 2.5 mM dNTP, 1 μl of Thermosequenase™ (usb), 1 μl of Thermosequenase™ Reaction Buffer (usb), 1.5 μl of pRc/CMV2-flag-VIPh and 1.5 μl of water were applied. Then 16 μl of the mixture was applied into four 0.2 ml tubes each and the same amount (16 μl) of ddATP, ddTTP, ddGTP and ddCTP were added respectively. PCR was performed with the solution mixture with standard procedure: firstly, 5 min at 95° C., followed by 30 cycles of 30 s at 94° C., 30 s at 50° C. and 50 s at 70° C., lastly, 50 s at 70° C.

In order for VIPt random fragment to be inserted, AflII linker was constructed. Oligo DNA fragment with AflII restriction site (AflII linker) was introduced into plasmid vector. It makes easy to insert VIPh and VIPt fragments into pRc/CMV2-flag sequentially. Two complementary oligonucleotide sequences of the linker were: 5′-CTA GAA GTC CTT AAG AGT CGG GCC-3′ (SEQ ID NO: 10; AflII linker 1) and 5′-CGA CTC TTA AGG ACT T-3′ (SEQ ID NO: 11; AflII linker 2). In the AflII linker, cohesive end suitable for XbaI and ApaI (GGGCCC) sites were formed in each 5′ and 3′ ends, respectively.

First, mixture of: 20 μl of AflII linker 1, 20 μl of AflII linker 2 and 0.5 μl of 1M Tris/HCl ph7.5 were boiled at 100° C., then cooled down to the room temperature in order to anneal to each other. Second, pRcCMV2-flag-VIPh vector was digested at its XbaI and ApaI restriction site by appropriate enzymes, then the vector was rescued by 1% agarose gel electrophoresis followed by QIAquick Gel Extraction. Third, annealed fragments of the linker and pRcCMV2-flag-VIPh (digested at XbaI site in 3′ end of VIPh, and ApaI site in pRc/CMV2) was ligated as follows: 3 μl of 32 nM pRc/CMV2-flag-VIPh, 3 μl of roughly 172 μM AflII linker, 6 μl of Solution I (Takara) of Takara ligation kit. Ligation mixture was incubated at 15° C. for 6 h or overnight.

Ligation mixture was then transformed into E. coli cells (JM109). In detail, 4 μl of the reaction mixture were added into 50 μl of CaCl₂ treated E. coli, chilled in 15 ml tube beforehand. It was kept on ice for 30 min, followed by incubation at 42° C. for 1 min without shaking the tube. Immediately it was cooled on ice for 2 min. After transformed cells were cultured with SOC medium (SOB medium supplemented with 10 ml of 2 M glucose; SOB medium: bacto trypton 10 g, bacto yeast extract 2.5 g, NaCl 0.29 g, KCl 0.09 g, 1 M MgCl₂+1 M MgSO₄ mix 10 ml, per 500 ml) at 37° C. for 1 h, cells were applied on a LB-Amp plate and incubated overnight at 37° C. for colonies to grow.

Four colonies were picked up and cultured in LB-Amp medium overnight, followed by plasmid extraction. Of these four colonies, at least one colony was confirmed to have plasmid by digestion with AflII followed by electrophoresis (FIG. 2C). Plasmid (pRc/CMV2-flag-VIPh-AflII) was sequenced and stored in 100 μl of TE buffer (350 ng/μl).

(2) VIPt Insertion Into pRc/CMV2-flag-VIPh-AflII

VIPran3 and VIPran5 primers were used in PCR to construct 3′ half of VIP36 gene (VIPt, 481-1071 nucleotides of the entire length of 1407 nucleotides, with mutations introduced) (FIG. 2A). A KOD dash DNA polymerase (TOYOBO), with a relatively loose proofreading activity, was chosen for PCR, since VIPₜ fragments were to include randomly mutated nucleotides. Various PCR conditions were tested to optimise reaction conditions, as numerous amount and wide diversity of mutated VIPt fragments were required for random library construction. As a result, the optimal concentration for the randomized primer (VIPran3 primer) was determined to 30 pmol/μl. In contrast, the concentration for the VIPran5 primer was determined to 10 pmol/μl. In particular, PCR containing 25 mM MgSO₄ was selected.

KOD plus (TOYOBO) DNA polymerase was also used for PCR amplification and some conditions were tested for optimisation.

VIPt fragments (FIG. 2B) were digested with AflII (3′ end of the fragments) and XbaI (5′ end). Eelctrophoresed to remove the small digested fragments, VIPt fragments were then ligated to pRc/CMV2-flag-VIPh-AflII, which XbaI site at the 3′ end of VIPh and AflII site in AflII linker of pRc/CMV2-VIPh-AflII were digested (FIG. 2D). Ligation solution was incubated at 15° C. overnight. Importantly, restriction site AflII was digested by restriction enzyme BfrI for two reasons. First, it recognised the same sequence as AflII enzyme did and, second, it was reported by the manufacturer that the site digested by AflII enzyme resulted in low efficiency of ligation reaction, which was critical for library construction.

Transformation into E. coli JM109 was done using a standard method as above.

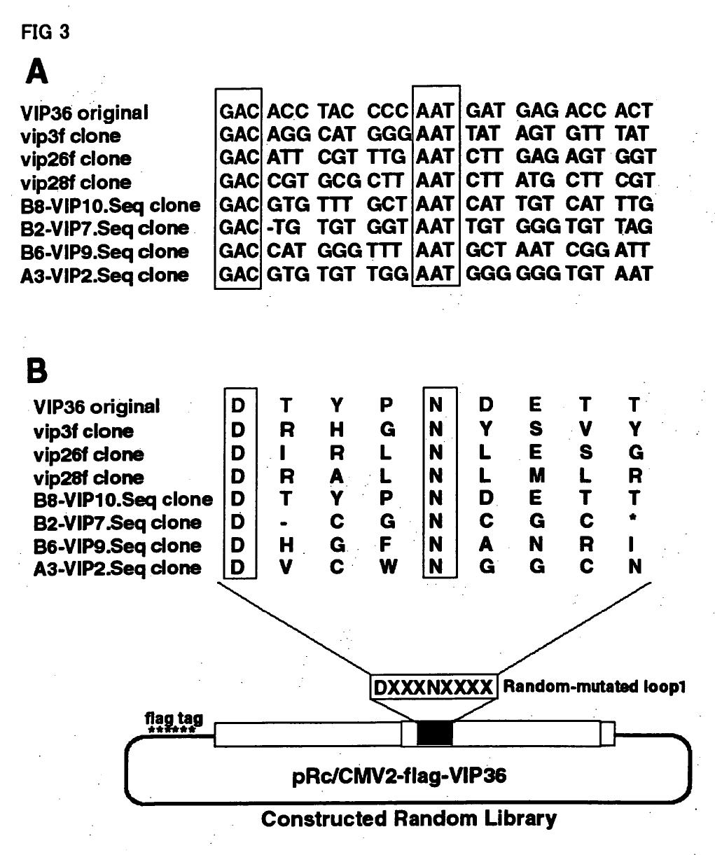

Electroporation was also performed using ElectroMax™ DH5alpha-E™ cells (Gibco BRL, Life Technologies: Catalog No. 11319-019). Before electroporation was performed, ligation solution was purified and concentrated by ethanol precipitation with standard protocol. Also, 1 ml of SOC medium in a 15 ml tube was pre-incubated at 37° C., a cuvette was chilled on ice, and 1 μl of pRc/CMV2-flag-VIPh-VIPt DNA in another fresh 15 ml was chilled on ice. Twenty μl of DH5alpha-E™ cells was applied in the above tube containing pRc/CMV2-flag-VIPh-VIPt DNA. After two times of gentle pipetting, plasmid-E. coli mixture was transferred to a previously chilled cuvette. Then electroporased with conditions of: 2.45 kV and 129 Ω by Electro Cell Manipulator® 600 (BTX). The above condition was chosen after a brief examination of several conditions in voltage. Voltages examined were 1.5, 2.0, and 2.45 kV. Immediately (within 1 min), 1 ml of pre-warmed SOC medium was added and mixed gently. Solution was then removed into a fresh 15 ml tube and incubated at 37° C. for 1 h. Then, each 100 μl of one-hour-cultured solution was spread onto a LB-Amp agar plate. Plates were then incubated overnight at 37° C. Electroporation was repeated until the number of colonies formed reached 1×10⁶. Some colonies were chosen for sequence analysis to confirm whether the random mutation of pRc/CMV2-flag-VIPh-VIPt was introduced (FIG. 3). Numbers of inserted fragment in a vector, transformed by both calcium chloride method and electroporation were determined by restriction digestion with XbaI and BfrI.

(3) Recovery of VIP36 Random Library

First of all, 50 ml of LB-Amp medium was added into a LB-Amp agar plate on which colonies were grown. Colonies were physically removed from the agar by gently mixing colonies with the added medium using a conradi stick (TGK). Suspension was then transferred to a fresh 50 ml tube. Suspension was then messed up to 50 ml with LB-Amp medium in order to give additional medium for cell growth. Suspension in medium were cultured overnight at 37° C. Air conditioning was noted while incubation. Five hundred μl of overnight cultured E. coli suspension was recovered as a stock, stored with 17% glycerol at −80° C. The above procedure was done with each single agar plate. In order to extract plasmids from the suspension, appropriate volumes of the suspension from usually ten different agar plates were mixed together. Volumes of the suspension to be mixed from each plate were determined according to the ratio of the number of colonies from each plate. This was to avoid for a particular mutated VIP36 DNA to predominate over the other ones. Plasmids were recovered from the mixture by QIAGEN Plasmid Midi Kit and stored in 100 μl of TE buffer. Then plasmids (mixture of pRc/CMV2-flag-VIPh-VIPt with putatively ramdomised carbodhydrate recognition domains in VIPt) were pooled in thirty-two 1.5 ml tubes and used in the following experiments.

To examine the mutated VIP36 cDNA to be introduced into plasmids, a few colonies were picked up and extracted plasmid from E. coli cells were digested with XbaI and BfrI. In a plate with low number of total colonies grown (usually from 40 to 200 colonies), 10 out of 12 colonies examined (83%) contained the target plasmid, whereas 4 out of 6 colonies (67%) had the target plasmid in a plate with high number of total colonies (from 1000 to 6000 colonies). Hence, the estimated size of the library was either 8×10⁵ or 6×10⁵. Randomly mutated gene encoding carbohydrate recognition domain of VIP36 was confirmed by nucleotide sequence analysis (FIG. 3).

### EXAMPLE 2

### Transfection Into Cells

(1) Stable Transfection by Effectene™

Because VIP36 library constructed in Example 1 was stored in thirty two 1.5 ml tubes, plasmids in 117.4 μl of TE buffer was first prepared by combining portions of library solutions separately stored in thirty two independent pools. Ratio of solutions mixed was determined according to the size of independent clones including in library of each pool. This calculation was to keep equal of the diversity of randomised carbohydrate recognition domain (CRD). In detail, 2.5 μl (0.5 μl×5 tubes) was taken from library of the size being 500 clones, 5.4 μl (0.6 μl×9) from library of the size 6000 clones, 0.1 μl (0.1 μl×1) from library of the size 1000 clones, 1.0 μl (1 μl×1) from library of the size 1×10⁴ clones, 66.0 μl (6 μl×11) from library of the size 6×10⁴ clones, 1.5 μl (1.5 μl×1) from library of the size 1.5×10⁴ clones, and 32.0 μl (8 μl×4) from library of the size 8×10⁴ clones.

The plasmid prepared as above (named VIP36mix) was linearized by digestion with BglII and incubated at 37° C. overnight. Linearized VIP36mix was then applied to 1% agarose gel electrophorasis and extracted from the gel. Concentration of the DNA after digestion was 475 ng/μl.

One day before transfection, Mardin-Darby canine kidney (MDCK) cells (ATCC accession number: CCL-34) were subcultured so that the cells were 80-90% confluent on the day of transfection.

According to procedures provided by the manufacturer, VIP36mix plasmid was transfected into MDCK cells. Four pg (i.e. 8.42 μl) of VIP36mix was applied into a 15 ml tube. Then 300 μl of EC buffer (QIAGEN, provided in a kit of Effectene transfection reagent) was added and mixed gently using voltex mixer for just one second. The mixture was left at room temperature for 4 min. Fourty eight μl of Effectene reagent (QIAGEN) was added to the above mixture and voltexed gently for 10 sec. The mixture was again left at room temperature for 9 min. While the preparation of DNA, D10 medium (Dulbecco's modified Eagle's medium (DMEM; Sigma) supplemented with 10% bovine fetal serum (FCS), 10 mM HEPES and penicillin (100 U/ml)-streptomycin (100 μg/ml)) was decanted and cultured MDCK cells in a plastic dish (φ100 mm) was washed with 5 ml of PBS (phosphate-buffered saline). The cells were suspended in 7 ml of fresh D10 medium. In the other tube, 3 ml of D10 medium was added to the DNA-reagent mixture prepared as above, mixed by gently pipetting 2 times. Immediately the mixture was applied drop by drop onto MDCK cells just prepared above. After the cells were cultured for 2 days at 37° C. in a 5% CO₂ incubator, D10 medium was replaced and G418 was added (final concentration: 2.0 mg/ml) for selection of successfully transfected cells. Cells were left for 14 days since transfection until neomycin-resistant cells grew. pRc/CMV2 vector has a neomycin-resistant gene which was expressed when transfected successfully. 14 days after transfection, approximately 100 colonies were formed in the presence of 2.0 mg/ml of G418.

(2) Stable Transfection by LipofectAMINE™ 2000

Same amount of VIP36mix was prepared as mentioned above. Cells were prepared two days before transfection so that they were over 95% confiuent when transfection was performed. Cells were cultured in 10 ml of D10 medium.

On the day of transfection, 24 μg of VIP36mix DNA (i.e. 55 μl of 439 μg/μl DNA) was diluted into 1.5 ml of OptiMEM® I Reduced Serum Medium (Gibco BRL). At the same time, 75 μl of LipofectAMINE™ 2000 Reagent (Invitrogen) was added into another 1.5 ml of OptiMEM® I Reduced Serum Medium and incubated at room temperature for 5 min. Then both VIP36mix DNA and LipofectAMINE™ 2000 Reagent in separate medium were combined and incubated at room temperature for 30 min. While VIP36mix DNA and LipofectAMINE™ 2000 Reagent were forming complexes, MDCK cells were washed with 5 ml of PBS two times and 13 ml of OptiMEM® I Reduced Serum Medium was added. When ready, 3 ml of the mixture of VIP36 DNA and LipofectAMINE™ 2000 Reagent was added directly into MDCK cells drop by drop. Plate was rocked back and forth. Cells were incubated for 24 h at 37° C. in a 5% CO₂ incubator.

After 24 h of incubation, OptiMEM® I Reduced Serum Medium was removed and subcultured, with standard passage culture protocol, in 22.5 ml of D10 medium in two 15 cm dishes. After 24 h later G418 was added into the two 15 cm dishes (final 2.0 mg/ml). Cells were incubated at 37° C. in a 5% CO₂ incubator for 10 days in order for G418-resistant cells to grow. 10 days after transfection, cells were about 80% confluent in the presence of 2.0 mg/ml of G418.

(3) Observation of Mutated VIP36 by Fluorescence Microscopy

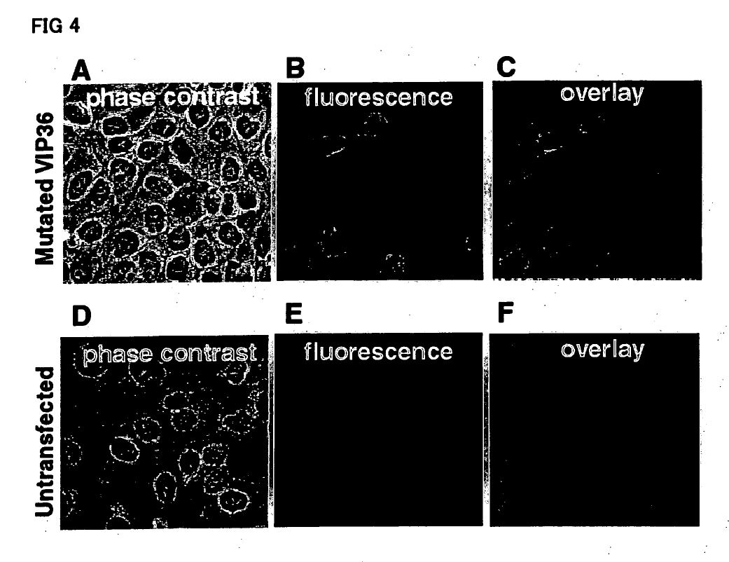

To estimate the transfection rates of MDCK cells, samples were prepared for fluorescence microscopy. Micro cover glasses (18×18 mm, MATSUNAMI) were sterilised in 100% ethanol and by a gas burner, and laid on the bottom of each well of a 6 well plate before cells were applied. After 4×10⁴ cells per sample were passaged into a 6 well plate for 24 h, cells were washed with PBS once. Then 1 ml of 4.0% formaldehyde in PBS was added and incubated at room temperature for 1 h to fix the cells. Fixed cells were washed by PBS once, they were treated with 1 ml of 0.3% TritonX-100 (Sigma) in PBS so that antibodies could permiabilize plasma membrane to bind intracellular antigens. Cells were then allowed to stand at room temperature for 20 min, followed by five-minutes-washing with PBS 3 times. Blocking was next performed by adding 1 ml of 5% BSA (bovine serum albumin) in PBS and placed at room temperature for 15 min. Cells were washed for 5 min with PBS three times. After PBS around a cover glass was removed by swabbing, 200 μl of anti-flag antibody (2 μg/ml) in 5% BSA/PBS, which was prepared from 3 mg/ml anti-flag M2 antibody (IBI), was mounted on a cover glass where cells were grown for observation. Cells were left at room temperature for 1 h for anti-flag antibody to bind flag tag of mutated VIP36. Washed for 10 min three times, blocking was done as described above. Following washing for 5 min by PBS three times, 200 μl of fluorescein isothyocyanate (FITC)-labelled anti-mouse IgG₁ (GAM-FITC, Molecular Probe, 10 μg/ml) in 5% BSA/PBS was applied on a cover glass and incubated for 1 h at room temperature for GAM-FITC to bind anti-flag antibody. Then cells were washed for 10 min with PBS three times. Lastly, 3 μl of 100 mM DAPI (4′,6-diamidino-2-phenylindole dihydrochloride, Molecular Probes) and 3 μl of COMPONENT A (antifade reagent in glycerol buffer) of Slowfade® Light Antifade Kit (Molecular Probes) was added on a micro slide glass (MATSUNAMI), and a prepared cover glass was placed on the slide glass, with cell-grown side of cover glass facing down to the slide glass. Sample was encapsulated by manicure (Kanebo) and observed by a fluorescent microscope (Olympus BX52, FIGS. 4A-F).

Among cells captured in microscopy, it was revealed that 50 of 792 cells (6.3%) had overexpressed mutated VIP36. The estimated number of transfected cells were, therefore, 1.26×10⁵ cells per 2.0×10⁶ cells. The number of 2.0×10⁶ was obtained by counting confluent cells on φ100 mm dish twice or three times. Representative photographs in phase contrast image (FIG. 4A or D), fluorescence image (FIG. 4B or E) and overlay of the both (FIG. 4C or F) were shown for both transfectant and wild type MDCK cells, respectively.

### EXAMPLE 3

### Carbohydrate Moiety-Based Separation of Transfected Cells

(1) Plant Lectins

Several plant lectins were used to separate transfected MDCK cells according to particular structure of oligosaccharides displayed on the cell surface. 10 kinds of lectins were used to distinguish a variety of oligosaccharides. Carbohydrate-binding specificity of lectins used were precisely analysed as shown in Table 1.

Biotinylated lectins (Honen Co.) were chosen so that lectin-bound cells could be recognised by Streptavidin MicroBeads (Miltenyi Biotec, colloidal paramagnetic MicroBeads conjugated to streptavidin). Lectins used were ABA lectin, ConA lectin, DSA lectin, LCA lectin, Lotus lectin, MAM lectin, Phaseolus vulgaris lectin with homotetrameric E-subunits (PHA-E₄), Phaseolus vulgaris lectin with homotetrameric L-subunits (PHA-L₄), RCA120 lectin, and WGA lectin. These lectins were selected among others because most of them were known to recognise N-linked and O-linked oligosaccharides. N-linked oligosaccharides were thought to be recognized by native VIP36 lectin. For magnetic cell sorting, lectins of 1 mg/ml were diluted in PBS (final 5 μg/ml) and sterilised by filtration using 25 mm Acrodisc® Syringe Filter 0.2 μm (Pall Co.).

(2) Magnetic Cell Sorting of Mutated VIP36 Transfected MDCK Cells

Magnetic cell sorting (MACS), the system that separates magnetically labelled cells from unlabelled cells, was chosen to collect cells based on carbohydrate moieties presented on the cell surface. MiniMACS, MACS separator for separation of cells and macromolecules, were purchased from Miltenyi Biotec. MiniMACS was suitable for separation of up to 10⁷ magnetically labelled cells, and a set of a MiniMACS Separation Unit, a MACS Mutli Stand, and MS Columns were included. Streptavidin MicroBeads (colloidal paramagnetic MicroBeads conjugated to streptavidin) was used for indirect labelling of biotinylated lectin-bound cells.

Buffers used in this experiment were Labeling buffer (1×PBS with 2 mM EDTA) and Separation buffer (1×PBS with 2 mM EDTA and 0.5% BSA) as described by the manufacturer. Both buffers were sterilised by filtration using 25 mm Acrodisc® Syringe Filter 0.2 μm. PBS-EDTA or Trypsin-EDTA was used to suspend the cells cultured on φ100 mm dish.

A day before separation, mutated VIP36 transfected MDCK cells were propagated so that about 80 to 90% confluent on the day of separation. Cells were washed with PBS twice and treated with either Trypsin-EDTA or PBS-EDTA for 20 min. After harvesting the cells were suspended in 2 ml of D10 medium to count the number of cells. Following centrifuge at 19 G (1000 rpm) for 5 min (same centrifugation condition applied to all the centrifuge below), cells were washed once by 10 ml of PBS. Centrifuged once again, cells were then diluted in 15 ml of lectin solution (5 μg/ml). After the incubation on ice for 30 min, lectin-bound cells were collected by centrifugation. Supernatant was aspirated carefully and completely. To wash cells for optimised secondary antibody binding, 300 μl (or 1 ml) of Labeling buffer was added. Cells were washed twice. Centrifuged once, 10 μl of Streptavidin MicroBeads was added to bind biotinylated lectins. Solution was mixed well and allowed to stand at 8° C. for 15 min. MicroBeads-bound cells were then collected by centrifuge, resuspended in 500 μl of Separation buffer and placed on ice for the following separation.

Before the separation process to begin, a MS column was prepared on MiniMACS Separation Unit clinging onto MACS Multi Stand, both sterilised by 70% ethanol.

In order to separate cells, a MS column was first prepared by adding 500 μl of Separation buffer. Then MicroBead-bound cells in 500 μl of Separation buffer were applied on the column. Applied solution was passed thourgh by gravity flow. The flow-through, which contain negative cells, was collected in a 15 ml tube. The column were washed thoroughly by 500 μl of Separation buffer three times. Separation buffer passed through the column in washing process was also collected in the same 15 ml tube. The MS column was then removed from MACS Multi Stand for positive cell recovery. Positive cells in the column was collected in a fresh 15 ml tube by addition of 1 ml of Separation buffer, immediately followed by firmly flushing out positive cells using the plunger provided by the manufacturer. Positively and negatively collected cells were counted by a hemocytometer (Erma) and shown in Tables 2A and 2B. In the tables, the number describing in the columns of transfected MDCK (A) and (B) indicates the result from two independent transfection experiments.

TABLE 2

| | Transfected | | Tansfected | | | |
|---|---|---|---|---|---|---|
| Types of Cells | MDCK (A) | | MDCK (B) | | Wildtype MDCK | |
| MACS fraction | positive | negative | positive | negative | positive | negative |

A: Numbers of cells separated by plant lectins [×10⁵].

| | | | | | | |
|---|---|---|---|---|---|---|
| ConA | 16 | 1.6 | 2.1 | 5.7 | 4.4 | 0.4 |
| LCA | 11 | 12 | 3.2 | 3.5 | 3.3 | 1.4 |
| PHA-E₄ | 0.8 | 22 | 3.5 | 0.55 | 3.3 | 1.6 |
| RCA120 | no cell | no cell | n/a | n/a | n/a | n/a |
| WGA | 13 | 24 | 5.2 | 6.2 | 2.3 | 1.65 |
| ABA | 9.2 | 18 | 5.8 | 12 | 4.3 | 0.85 |
| PHA-L₄ | 16 | 9.2 | 1.6 | 2.1 | 1.85 | 2.95 |
| MAM | 17 | 20 | 0.38 | 22 | 0.85 | 4.55 |
| Lotus | n/a | n/a | 0.68 | 23 | 0.2 | 4.95 |
| DSA | n/a | n/a | 5 | 21 | 4.05 | 0.6 |

B: Ratio of positive and negative cells separated by plant lectins (%).

| | | | | | | |
|---|---|---|---|---|---|---|
| ConA | 96.4 | 9.1 | 26.9 | 73.1 | 91.7 | 8.3 |
| LCA | 47.8 | 52.2 | 47.8 | 52.2 | 70.2 | 29.8 |
| PHA-E₄ | 3.5 | 96.5 | 86.4 | 13.7 | 67.3 | 32.7 |
| RCA120 | n/a | n/a | n/a | n/a | n/a | n/a |
| WGA | 35.1 | 64.9 | 45.6 | 54.4 | 58.2 | 41.8 |
| ABA | 33.8 | 66.2 | 32.6 | 67.4 | 83.5 | 16.5 |
| PHA-L₄ | 63.5 | 36.5 | 43.2 | 56.8 | 38.5 | 61.5 |
| MAM | 45.9 | 54.1 | 1.7 | 98.3 | 15.7 | 84.3 |
| Lotus | n/a | n/a | 2.9 | 97.1 | 3.9 | 96.1 |
| DSA | n/a | n/a | 19.2 | 80.8 | 87.1 | 12.9 |

All the procedures mentioned above applied to each lectin of ten examined: ConA, LCA, PHA-E₄, WGA, ABA, PHA-L₄, MAM, RCA120, Lotus and DSA lectins. For first 7 lectins experiment was independently carried out twice.

Separated cells were collected as lectin-positive or -negative fractions. The numbers of recovered cells in both positive and negative fraction were counted by a hemocytometer (Table 2A) and the ratio of separation was shown in Table 2B. Calculated ratios showed that separation ratio of PHA-E₄ in the first experiment was reversed for the second time (third row in Table 2B). Ratio of positive fraction by MAM was decreased in the second experiment from 45.5% of total cells recoverd to 1.7% (eighth row of Table 2B). In comparison with separation ratios of wild type MDCK, positive fraction by Lotus resulted in 2.9 and 3.9% of total cells recovered in separation of both transfectant and wild type MDCK cells surface (second row from the bottom of Table 2B). It might suggest few fucose residues are displayed on the cell. DSA lectin separation showed less positively recovered cells of transfectants than that of wild type cells (the bottom row of Table 2B)).

At the time of first experiment was carried out (result of (A) in Tables 2A and 2B), PHA-E₄ lectins were chosen as its positive fraction shared 3.5% of the total recovered. Carbohydrate-binding specificity of the positive fraction was observed also by flow cytomeric anlysis (see Example 4).

(3) Multiple Magnetic Cell Sorting of PHA-E₄ and WGA Positive Transfected Cells

Two groups of mutated VIP36 transfected MDCK cells were proceeded to second and third round of magnetic cell sorting for larger extent of separation of each cell group than the separation method of the above (2). One group of transfected cells chosen were PHA-E₄ lectin positive group, the other was WGA positive one. These two groups of cells were further applied to sorting by MiniMACS three times with procedures described above, and the former and the latter enriched the positive cells and negative cells, respectively. Similarly, cells were subjected to further magnetic cell sorting three times. Both group of cells were counted by a hemocytometer (Table 3)

TABLE 3
The number of cells sorted by PHA-E₄ and WGA [×10⁵].

| No. of | MiniMACS, | | MiniMACS, | | MiniMACS, | |
|---|---|---|---|---|---|---|
| MiniMACS | 1^{st} round | | 2^{nd} round | | 3^{rd} round | |
| MACS fraction | positive | negative | positive × 2 | negative × 2 | positive × 3 | negative × 3 |
| PHA-E₄ | 0.8 | 2.2 | 4.5 | 4 | 3.8 | 10.6 |
| WGA | 5.2 | 6.2 | 4.8 | 4.2 | 6.7 | 1.1 |

(4) Magnetic Cell Sorting of Wild Type MDCK Cells

Wild type of MDCK cells were examined by MiniMACS with procedures described above. Cells were counted as above and shown in Table 2. Biotinylated lectins used were: ConA, LCA, PHA-E₄, WGA, ABA, PHA-L₄, MAM, Lotus and DSA lectins.

### EXAMPLE 4

### Cell Surface Glycosylation Analysis by Flow Cytometry

(1) Flow Cytometry of Separated Cells of Both Transfectant and Wild Type MDCK Cells

In this example, in order to investigate the carbohydrate structures on the cells accompanying with overexpression of mutated VIP36 gene, flow cytometric analysis was performed. Fluorescence intensity of the cells, stained with biotinylated lectin followed by streptavidin-FITC, was measured and recorded in histograms. Transfected cells positively separated once, twice or three times by MiniMACS, and transfectant negatively separated once, twice or three times were subjected to these analyses. Two groups of wild type MDCK cells separated positively or negatively by MiniMACS were also analysed to be compared with those of transfectants. In total, 53 independent groups of MDCK cells prepared by MiniMACS separation were used. Lectin-positive and -negative groups (2 groups) of both transfected and wild type MDCK cells (2 cell types) for each lectins (9 lectins; ABA, ConA, DSA, LCA, Lotus, MAM, PHA-E₄ and WGA) were subtotal of 36 groups. Enriched cell groups obtained by multiple magnetic sorting made subtotal of 8 groups. Control MDCK cells for 9 lectins were subtotal of 9 groups.

On the day before flow cytometric analysis, each group of MDCK cells was cultured in 4 ml of D10 medium in a 6 cm dish so that cells did not reach confluency when analysed.

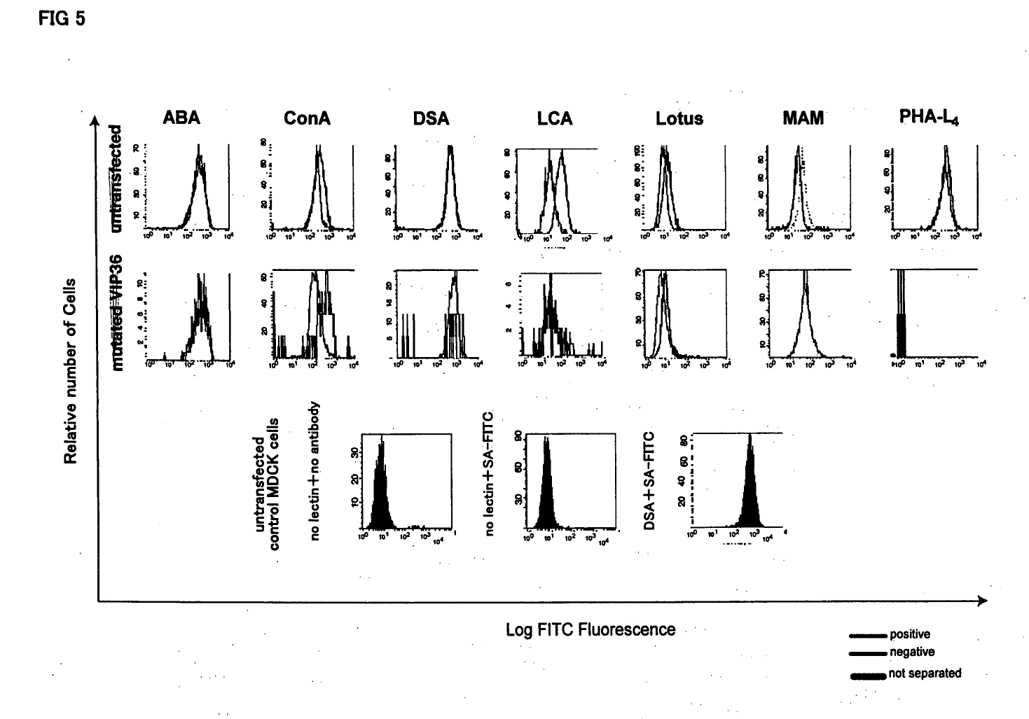

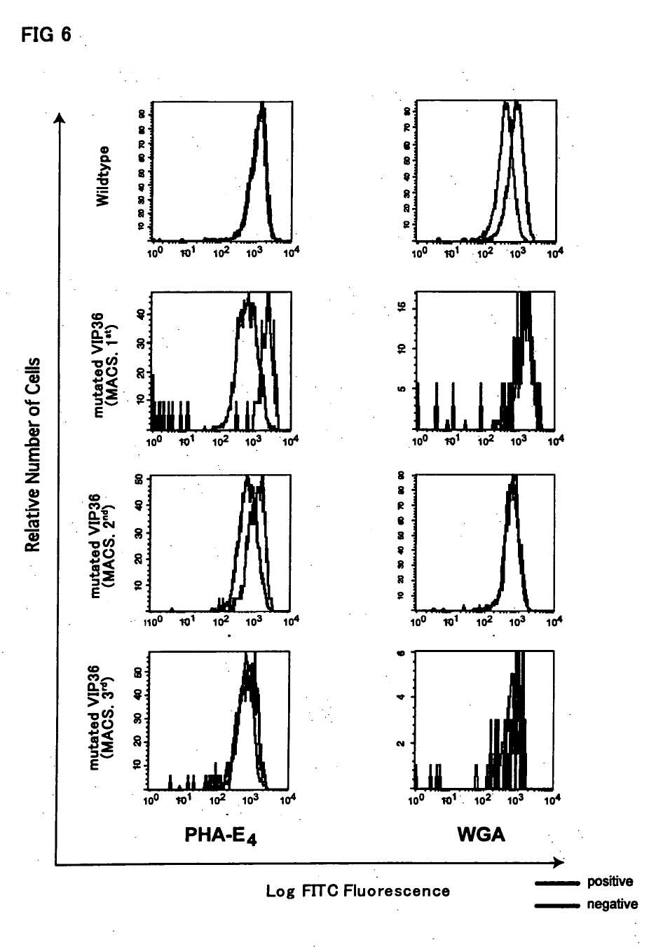

On the day of analysis, cells in each dish was washed twice with PBS, followed by treatment with 500 μl of PBS-EDTA (or Trypsin-EDTA) at 37° C. for 30 min in a 5% CO₂ incubator. When suspended, 250 μl of cells were transferred to 96 well plate and centrifuged at 51 G (1800 rpm) for 1 min (same condition applied to all centrifuge mentioned below). Then cells were washed by 200 μl of FACS buffer (HBSS (Gibco BRL) containing 0.1% BSA and 0.1% sodium azide). For primary staining, 50 μl of each lectin (10 μg/ml) was added to each appropriate well, followed by incubation on ice for 30 min. After lectin binding reaction, washing by 100 or 200 μl of FACS buffer followed by centrifuge and decantation was done three times. Decantation of FACS buffer was carefully carried out by quickly turning the 96 well plate upside-down to avoid contamination. Secondary antibody binding was performed by addition of 50 μl of streptavidin-FITC (20 μg/ml) with incubation on ice for strictly 30 min. Following washing twice by 150 or 200 μl of FACS buffer, each cell pellet was suspended in 200 μl of FACS buffer and removed to a 1.2 ml tube (Marsh Biomedical Products). After addition of 100 μl of 3 μg/ml propidium iodide (PI, Sigma), cells were analysed by FACScalibur® (Becton Dickson). Data analysis in a computer was carried out with CellQuest® (Becton Dickson) programme. The results are shown in FIGS. 5 and 6.

In case of mutated VIP36 transfected cells, most of separated cells showed no significant differences in separation ratio for the first time experiment. This result was supported by flow cytometric analysis using the same lectins (FIG. 5). In this example, separation of cells using MACS was not so good for lectins that show slight difference in the percentage of negative cells and positive cells shown in Table 3 above. Thus, cells were well sorted using FACS as described in Example 12. PHA-E₄+ and PHA-E₄− cells were, however, separated with apparently unique separation ratio even when using MACS. Mean values of relative fluorescence intensity measured was 1786.00 for TF-PHA-E₄+, 649.30 for TF-PHA-E₄−, whereas 1034.20 and 1127.68 for the wild type, respectively. The mean value of non-separated wild type MDCK cells was 852.76. In the specification, “TF” means a fraction sorted from the transfected cells. Both TF-PHA-E₄+ and TF-PHA-E₄− cells were further enriched by MiniMACS separation, and analysed by flow cytometry. Results showed that the mean values of TF-PHAE4++ (positive cells collected after two MiniMACS separation, same abbreviation applies below) and TF-PHAE4−− were 1135 and 660, respectively (FIG. 6).

(2) Effect of Treatment by PBS-EDTA or Trypsin-EDTA

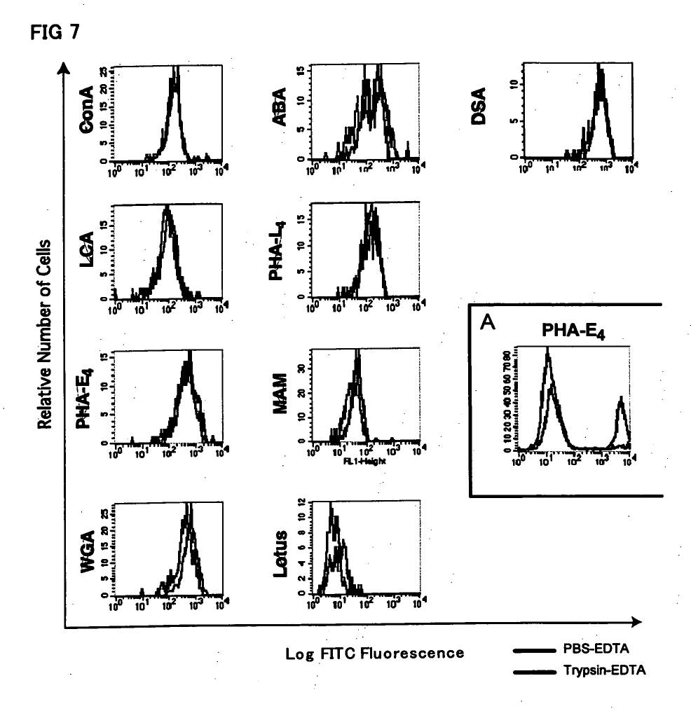

In order to examine the effect of trypsin on cell surface glycosylaiton, two independent groups of wild type MDCK cells were treated with PBS-EDTA or Trypsin-EDTA, respectively, when cells were suspended for primary staining. Flow cytometry procedures were proceeded as mentioned above (1). The results are shown in FIG. 7.

Out of 9 lectins used, 8 lectins showed no significant difference in binding affinity, whereas cells stained with Lotus lectin displayed higher binding affinity of trypsin treated cells (FIG. 7). In addition, it was noted that trypsin treated cells were hardly stained with PI, suggesting most of cells did not die during experiments (in the lower box of FIG. 7).

In Examples 1-4 above, the experiments that the carbohydrate structures on the cell surface was modified by introducing random mutations into carbohydrate recognition domain of VIP36, which involves transport of glycoprotein in secretary pathway.

By using lectins in flow cytometry, it was demonstrated that MDCK cells had a variety of oligosaccharides on the cell surfaces. Transfected MDCK cells sorted by PHA-E₄ lectin clearly showed a significant difference in lectin-binding activity. Also, repeated screening using lectins may enrich cells clone of which specificity for a certain carbohydrate moiety is enhanced.

### EXAMPLE 5

### Design of PCR Primer for Introducing Random Mutation for ERGIC-53

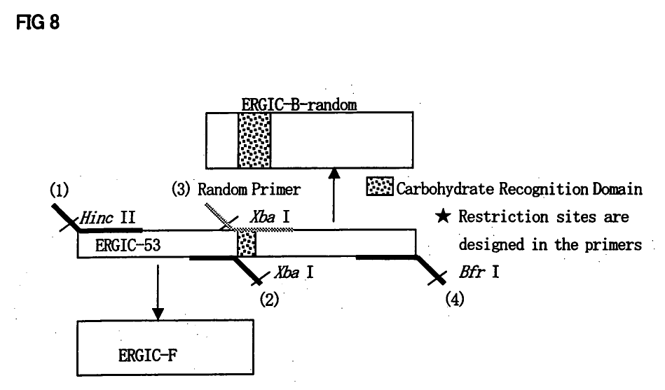

To introduce random mutation into a putative carbohydrate-binding domain of ERGIC-53 cDNA, the PCR method, by which introduction is carried out in two separate steps as in the case of VIP36 described in Example 1, was employed. FIG. 8 shows the correlation between designed primers and ERGIC-53. “(3)” was designed as a random primer for randomizing the putative carbohydrate-binding domain. PCR was performed using primers (1) and (2) and ERGIC-53 as a template. The thus generated DNA fragment (the first half portion not including the carbohydrate-binding domain of ERGIC-53, hereinafter referred to as ERGIC-F) was incorporated into pRC-CMV2-CD8-FLAG (a vector prepared by ligating a CD8 signal sequence and a FLAG tag to pRC-CMV2). Subsequently, PCR was performed using the random primer (3) and a primer (4) and ERGIC-53 cDNA as a template. The thus generated DNA fragment (the latter half portion including the carbohydrate-binding domain of ERGIC-53, hereinafter referred to as ERGIC-B-random) was introduced into the pRC-CMV2-CD8-FLAG wherein the ERGIC-F had been inserted. With such a technique, an ERGIC-53 random library was constructed. Since the region into which random mutations had been introduced by this technique is not amplified by PCR, various mutations can be introduced evenly.

The names and the nucleotide sequences of the prepared primers are as shown below:
(1) ERGIC-FF (underlined portion corresponds to Hinc II site)
   5′-GTACGTCGACGGCGTGGGAGGAG-3′ (SEQ ID NO: 12)
(2) ERGIC-FR (underlined portion corresponds to Xba I site)
   5′-CGTATCTAGAAATATTCCAACACCATTCCA-3′ (SEQ ID NO: 13)
(3) ERGIC-BF (underlined portion corresponds to Xba I site; K corresponds to G

or T; X corresponds to any one of A, T, G, or C)

(SEQ ID NO: 14)

5′-CGTATCTAGATXXKXXKXXKAATXXKXXKXXKXXKAATAATCCTG
CTATAGTAATTAT-3′

(4) ERGIC-BR (underlined portion corresponds to Bfr I site)
   5′-CGTACTTAAGTGGTAGTCAAAAGAATTTTTTG-3′ (SEQ ID NO: 15)

### EXAMPLE 6

### Construction of ERGIC-53 Random Library

(1) Preparation of Plasmid pRC/ERGIC-F

The PCR method was performed using ERGIC-53 cDNA incorporated into pBluescript as a template, thereby amplifying the ERGIC-F gene, the first half portion not including the carbohydrate recognition domain of ERGIC-53. As primers, those designed in Example 5 above were used. ERGIC-FF was used as a forward primer and ERGIC-FR was used as a reverse primer. A PCR reaction solution was prepared by mixing 2 μl of the forward primer ERGIC-FF, 2 μl of the reverse primer ERGIC-FR, 2 μl of the template ERGIC-53 cDNA (1 ng/μl), 3.2 μl of 25 mM MgSO₄ (TOYOBO), 4 μl of 2 mM dNTPs (TOYOBO), 4 μl of 10×PCR buffer for KOD-PLUS (TOYOBO), 0.8 μl of KOD-PLUS DNA polymerase (TOYOBO), and 22 μl of Milli-Q water. PCR reaction was performed under conditions of 94° C. for 2 minutes, (94° C. for 15 seconds, 52° C. for 30 seconds, and 68° C. for 1 minute)×30 cycles, and 68° C. for 7 minutes. In addition, both primers that had been previously phosphorylated were used. From the PCR product, the target fragment ERGIC-F was separated by 1% agarose electrophoresis (Mupid-21 mini gel electrophoresis bath: COSMO BIO co, ltd.) and then purified by a Gene Clean Spin Kit (BIO101), so that insert fragments were prepared.

pBluescript II SK (+) was treated with a restriction enzyme Sma I (TOYOBO), dephosphorylated (treated with alkaline phosphatase (BAP) derived from Escherichia coli), followed by phenol/CHCl₃ extraction and ethanol precipitation, thereby obtaining a product to be used as a vector.

The above vector pBluescript II SK(+) that had been cut with Sma I and the DNA fragment ERGIC-F were mixed at a molar ratio of 1:10, followed by ligation. After ligation, the reaction solution was transformed into competent cells JM109 by the CaCl₂ method, and then the cells were inoculated on LB-Amp plates.

From among blue and white colonies that grew on the plates, only several white colonies were picked up and cultured overnight in 3 ml of an LB liquid medium (containing ampicillin at a final concentration of 100 μg/ml). Plasmids were then extracted by an alkaline prep method and then subjected to insert check. To colonies for which the insert DNA fragment had been found to exist in the vector, plasmid extraction was carried out again using a Plasmid Mini Kit (QIAGEN). Thus, the sequence of the insert DNA fragment was confirmed by a DNA sequencer (LI-COR).

3 out of 6 obtained DNA fragments could be confirmed to have correct nucleotide sequences. One of the fragments was determined to be a target vector to be obtained in this example, and then used in the following experiments.

Plasmids wherein the inserted DNA fragment having the correct sequence in the vector were selected, treated with restriction enzymes Hinc II (TOYOBO) and Xba I (TOYOBO), and then subjected to 1% agarose gel electrophoresis. Then, a target DNA fragment ERGIC-F was purified using a QIAquick Gel Extraction Kit (QIAGEN).

pRC-CMV2-CD8-FLAG (a vector prepared by ligating a CD8 signal sequence and a FLAG tag to pRC-CMV2) was treated with restriction enzymes Hpa I (TOYOBO) and Xba I (TOYOBO), subjected to 1% agarose gel electrophoresis, and then purified using a QIAquick Gel Extraction Kit (QIAGEN).

The above vector pRC-CMV2-CD8-FLAG (that had been cut with Hpa I and Xba I) and the above DNA fragment ERGIC-F were mixed at a molar ratio of 1:10, followed by ligation. After ligation, the reaction solution was transformed into competent cells JM109 by the CaCl₂ method, and then the cells were inoculated onto LB-Amp plates.

Several colonies were picked up from colonies that grew on the plates, and then subjected to insert check by the colony PCR method. As a result, those revealed to contain the insert were target pRC-CMV2-CD8-FLAG vectors having ERGIC-F incorporated therein. Hereinafter, the target vectors were referred to as pRC/ERGIC-F. For use in the following experiments, pRC/ERGIC-F was purified using a Plasmid Mini Kit (QIAGEN). Furthermore, glycerol stocks of pRC/ERGIC-F were prepared and stored in a refrigerator at −80° C.

(2) Preparation of pRC/ERGIC-Random

In a manner similar to the method described in (1) above, the PCR method was performed using ERGIC-53 cDNA as a template, thereby amplifying the latter half including the carbohydrate recognition domain of ERGIC-53, the gene of ERGIC-B-random. ERGIC-BF was used as a forward primer and ERGIC-BR was used as a reverse primer. PCR reaction was performed under conditions of 94° C. for 2 minutes, (94° C. for 15 seconds, 54° C. for 30 seconds, and 68° C. for 1 minute)×30 cycles, and 68° C. for 7 minutes.

The target fragment ERGIC-B-random was separated, and then purified using a QIAquick Gel Extraction Kit (QIAGEN).

Subsequently, the fragment was treated with restriction enzymes Xba I (TOYOBO) and Bfr I (TOYOBO), subjected to 1% agarose gel electrophoresis, and then purified using a QIAquick Gel Extraction Kit (QIAGEN). Thus an insert DNA fragment was obtained.

The vector pRC/ERGIC-F prepared in (1) above was treated with restriction enzymes Xba I (TOYOBO) and Bfr I (TOYOBO) and subjected to 0.8% agarose gel electrophoresis, thereby separating a target fragment. The fragment was then purified using a QIAquick Gel Extraction Kit (QIAGEN). Thereafter, the fragment was used as a vector.

The above vector pRC/ERGIC-F (that had been cut with Xba I and Bfr I) and the insert DNA fragment ERGIC-B-random were mixed at a molar ratio of 1:6, followed by ligation. After ligation, the reaction solution was transformed into competent cells JM109 by the CaCl₂ method, and then the cells were inoculated onto LB-Amp plates.

24 colonies were randomly picked up from colonies that grew on the plates, and then cultured overnight in 3 ml of an LB liquid medium (supplemented with ampicillin at a final concentration of 100 μg/μl), followed by plasmid extraction by an alkaline prep method. Subsequently, the plasmids were cleaved with 2 restriction enzymes Apa I (TOYOBO) and Hind III (TOYOBO), and then subjected to 1% agarose gel electrophoresis to confirm the presence of ERGIC-random in the plasmids. Inserts were contained in 21 out of 24 μplasmids picked up.

As a result of insert check, plasmids confirmed to contain the insert DNA fragment (ERGIC-B-random) were subjected to plasmid extraction (Plasmid Mini Kit (QIAGEN) was used) again. To confirm whether or not a portion corresponding to the insert DNA fragment (ERGIC-B-random) introduced in pRC/ERGIC-F had been reliably randomized by PCR, DNA sequence analysis was carried out. The nucleotide sequence to be analyzed at this time was the putative carbohydrate-binding domain of ERGIC-53, and was as short as 9 amino acids in length. Thus, the sequence was analyzed using an ABI sequencer. As a result of sequence analysis, the nucleotide sequences of the putative carbohydrate-binding domains were found to differ in all the plasmids.

(3) Construction of ERGIC-53 Random Library

In (2) above, pRC/ERGIC-Random (more specifically, the vector pRC-CMV2-CD8-FLAG, wherein ERGIC-53 having random mutations into the putative carbohydrate-binding domain) was prepared. Among 9 amino acids (DTFDNDGKK) of the putative carbohydrate-binding domain, 7 amino acids were randomized (DXXXNXXXX; X denotes any amino acid). However, in the present invention, there had been a need to produce many of those having various amino acids at the putative carbohydrate-binding domain. Hence, a pRC/ERGIC Random library was constructed. In addition, such a library is hereinafter referred to as an ERGIC random library.

(3-1) Examination of Conditions for the Construction of ERGIC Random Library

When the vector pRC/ERGIC-F prepared in (1) above and the insert DNA fragment ERGIC-B-random prepared in (2) above were ligated, the molar ratio of the vector to the insert was determined at 4 stages to be: 1:2, 1:3, 1:6, and 1:10. The ligated product was then transformed into competent cells JM109. In addition, the vector (approximately 6 Kb) with a concentration of 265 ng/μl and the insert (approximately 1 Kb) with a concentration of 520 ng/μl were used.

Upon transformation, the amount of DNA (after ligation) to be transformed into competent cells was varied: 2 μl, 3 μl, and 5 μl relative to 50 μl of competent cells.

2 types of competent cells (JM109 and DH5α) were used for transformation and then compared for efficiency (both types of cells were competent cells for use in the CaCl₂ method). In addition, the molar ratio of the vector to the insert upon ligation was 1:6.

As a result of the experiment, the number of colonies that had grown was the largest in the case where the molar ratio of the vector to the insert was 1:10. Thus, a molar ratio of the vector to the insert of 1:10 was determined to be an optimum molar ratio. Furthermore, when the amount of DNA was examined, in the case of 5 μl of DNA per 50 μl of competent cells, the number of colonies was largest. Hence, in this experiment, it was determined to use 5 μl of DNA that had been subjected to ligation per 50 μl of competent cells. Furthermore, the number of colonies that grew in the case where DH5a had been used was 5 times greater than that in the case where JM109 had been used. Thus, it was determined to use DH5α.

(3-2) Collection of ERGIC Random Library

The number of colonies that grew on the LB-Amp plates were counted for each plate.

24 colonies were randomly picked up for confirmation from colonies that grew on the plates, the number of which had been counted. These colonies for confirmation were cultured overnight in 3 ml of an LB liquid medium (containing 100 μg/ml ampicillin), followed by plasmid extraction and insert check (treated with restriction enzymes Apa I and Hind III, and then subjected to 1% agarose gel electrophoresis). 6 plasmids were randomly picked up from the plasmids confirmed to contain the insert, and then subjected to sequence confirmation (to confirm whether or not random mutations had been introduced into the carbohydrate-binding domain) by a DNA sequencer. Based on the results of insert check and DNA sequencing, the ratio of effective clones to clones that had grown on the plates was calculated.

Colonies other than those for confirmation were immediately collected. Among the collected colonies, colonies in an effective number as calculated in the above method were used for ERGIC random libraries. Specific methods for collection will be described as follows.

15 ml of an LB liquid medium (containing 100 μg/ml ampicillin) was added to each LB-Amp plate, and then colonies were physically peeled off using a conrage stick, followed by shake-culture overnight at 37° C. Subsequently, a part (400 μl) of the culture solution per plate was stored as glycerol stock. The remaining culture solution was subjected to plasmid extraction using a Plasmid Mini Kit (QIAGEN). In addition, plasmid extraction was carried out for several plates together. At this time, the culture solutions were mixed in an amount proportional to the number of colonies that had grown on each plate (when a culture solution collected from a plate on which 1000 colonies had grown was mixed with a culture solution collected from a plate on which 100 colonies had grown, the amount of the former culture solution was used in an amount 10 times greater than that of the latter culture solution, and then subjected to plasmid extraction).

The number of colonies collected was approximately 238,000. When colonies for confirmation were randomly selected at the time of collection and then subjected to insert check and DNA sequencing for confirmation, the inserts were contained in 20 colonies per 24 colonies on average. Regarding plasmids confirmed by DNA sequencing, the putative carbohydrate-binding domains have different nucleotide sequences. Hence, the number of effective colonies was determined to be approximately 200,000, representing 20/24 of thee collected 238,000 colonies. Thus, the size of each ERGIC random library constructed at this time was 2×10⁵ For these libraries, a glycerol stock was prepared for each plate, and then stored in a refrigerator at −80° C. Furthermore, approximately 5000 colonies mixed together were subjected to plasmid extraction, and then stored at −20° C. In the following examples, these plasmids were used in experiments.

### EXAMPLE 7

### Expression of ERGIC Random Library Using MDCK Cell

In this example, the ERGIC random libraries constructed in Example 6 were transfected into MDCK cells and then expressed. Subsequently, expression was confirmed by the Western blotting method (Example 8) and the indirect fluorescence antibody method (Example 9).

(1) Examination of Transfection Efficiency

Before transfection of the ERGIC random libraries into MDCK cells, conditions therefor were examined. Specifically, pIRES-EGFP (CLONTECH) was transfected into MDCK cells using an Effectene transfection reagent (QIAGEN) and an Lipofectamine™ 2000 reagent (Invitrogen), so that EGFP genes were expressed temporarily. Subsequently, transfection efficiency was examined by measuring fluorescence intensity using a fluorescence-activated cell sorting system (hereinafter referred to as FACS).

(1-1) Examination of Efficiency Using Effectene Transfection Reagent

The expression efficiency of an Effectene transfection reagent (QIAGEN) was examined using 0.526 μg/μl pIRES-EGFP (CLONTECH).

Specifically, 4×10⁵ cells were inoculated on a 6-well plate (FALCON 3046) on the day before transfection, and then cultured overnight at 37° C. in 5% CO₂. On the day of transfection, a medium (mixture of 500 ml of DMEM (SIGMA), 55.5 ml of immobilized fetal bovine serum (FBS; INTERGEN), 5 ml of 10 mM HEPES, and 5 ml of penicillin (100 U/ml)-streptomycin (100 μg/ml)) was aspirated from the plate. The cells were washed once with 1×PBS (1 ml), and then 1.6 ml of a new medium was added ((1)).

In the meantime, plasmid DNA was added to a 15-ml tube and then adjusted to be a total of 100 μl using a buffer EC. An Enhancer (QIAGEN, attached to an Effectene transfection reagent kit) was added to the solution. The solution was agitated using a Vortex mixer for 1 second, left to stand at room temperature for 2 to 5 minutes, and then spun down. Next, an Effectene transfection reagent was added to the solution. The solution was vortexed for 10 seconds, and then left to stand at room temperature for 5 to 10 minutes. 600 μl of a medium was added to the solution, and then pipetting was carried out ((2)).

The pipetted solution (2) was added to the solution (1) above, and then the mixture was cultured at 37° C. for 48 hours. When selection was carried out using a drug, the drug was added 48 hours after transfection.

Furthermore, at 48 hours after transfection, expression efficiency was compared by FACS. Specifically, cells at 48 hours after transfection were washed twice with PBS, 2 ml of PBS-0.5 mM EDTA was added thereto, and then the cells were cultured at 37° C. in 5% CO₂, so that the cells were released. When the cells were released, 2 ml of a FACS buffer (9.8 g of HBSS (Hanks' Balanced Salt Solution, NISSUI PHARMACEUTICAL CO., LTD.) was dissolved in Milli-Q, and then to which 0.35 g of NaHCO₃, 10 ml of 10% NaN₃, and 5 ml of 20% BSA/NaN₃ solution were added, followed by adjustment of the solution to 1 litter) was added. The solution was transferred into a 15-ml tube, and then centrifuged at 190×g for 10 minutes. The supernatant was discarded. Furthermore, the cells were washed once with 2 ml of a FACS buffer, the number of cells was determined, the solution was centrifuged at 190×g for 10 minutes, and then the supernatant was discarded.

The solution was diluted in a FACS buffer to achieve 8×10⁶ cells/ml, and then the diluted solution was transferred into a FACS tube. 3 μg/ml propidium iodide (SIGMA) was added in an amount half that of the diluted solution containing the cells, and then dead cells were stained. The cells were analyzed by CellQuest (Becton-Dickinson Immunocytometory Systems) using FACS Calibur. Viable cells were selected based on forward scattered light, lateral scattered light, and staining with propidium iodide (SIGMA). Thus, information on 10000 viable cells was collected and analyzed. In addition, fluorescence of GFP (green fluorescence protein) of introduced pIRES-EGFP (CLONTECH) was detected and analyzed at this time.

As a result, it was revealed that the best efficiency of transfection was 3.65% when the amount of a reagent containing 0.8 μg of DNA, 6.4 μl of an Enhancer, 8 μl of an Effectene transfection reagent, and 99.5 μl of a buffer EC was used.

(1-2) Examination of Efficiency Using Lipofectamine™ 2000 Reagent

Expression efficiency was examined for an Lipofectamine™ 2000 reagent (Invitrogen) using a pIRES-EGFP (CLONTECH, 4 μg/minute) with a concentration of 0.526 μg/μl.

Specifically, 4×10⁵ cells were inoculated on a 6-well plate (FALCON 3046) on the day before transfection, and then cultured overnight at 37° C. in 5% CO₂. The cells were grown to be 90% to 95% confluent on that day.

In the meantime, 4 μg of plasmid DNA was diluted in 250 μl of an Opti-MEM (attached within a Lipofectamine™ 2000 reagent kit, Invitrogen) ((1)). 10 μl of an Lipofectamine™ 2000 reagent was diluted in 250 μl of an Opti-MEM, and then cultured at room temperature for 5 minutes ((2)). DNA-Lipofectamine™ 2000 reagent complexes were formed by mixing solution (1) and solution (2) and then subjecting the resulting solution to static culture at room temperature for 20 minutes. The complexes were added to the cells, and then cultured at 37° C. in 5% CO₂ for 24 hours.

24 hours after transfection, media were exchanged with fresh media, followed by another 24 hours of culture. When selection was carried out using a drug, the drug was added at 48 hours after transfection.

Furthermore, at 48 hours after transfection, FACS analysis was carried out in a manner similar to that in (1-1) above.

As a result, the transfection efficiency was 8.20%. Therefore, it was determined to carry out the following transfection using the Lipofectamine™ 2000 reagent (Invitrogen).

(2) Expression of ERGIC Random Library

In this example, the ERGIC random libraries constructed in Example 6 were transfected into MDCK cells using an Lipofectamine™ 2000 reagent (Invitrogen). At this time, the ERGIC random libraries linearized by restriction enzyme treatment were transfected.

All the ERGIC random libraries (2×10⁵) constructed in Example 6 were subjected to restriction enzyme treatment with Sca I, and then purified using a QIAquick Gel Extraction Kit (QIAGEN). In addition, the ERGIC random library used herein was prepared by mixing colonies so as to contain all the colonies at the same ratio based on a calculation (in the case of plasmids obtained by collecting 500 colonies and plasmids obtained by collecting 5000 colonies, the concentrations thereof were each measured and then the latter plasmids were used such that the amount of DNA thereof was I0 times greater than that of the former plasmids). After purification, each concentration was measured using a spectrophotometer (JASCO, V-550).

4×10⁵ MDCK cells were inoculated on a 6-well plate (FALCON, 3046) on the day before transfection, and then cultured overnight at 37° C. in 5% CO₂. The ERGIC random library (which had been cut with Sca I) was transfected into the above cells using 4 μg of a Lipofectamine≯ 2000 reagent (Invitrogen) per well, and then the cells were cultured for 24 hours at 37° C. in 5% CO₂.

24 hours after transfection, media were exchanged with fresh media, followed by another 24 hours of culture at 37° C. in 5% CO₂.

48 hours after transfection, media were exchanged with media containing 1.5 mg/ml G418, and then selection was initiated. After the start of selection, the cells were observed once a day, so as to confirm how the selection had proceeded.

10 days after the initiation of selection, cells that had grown in media containing 1.5 mg/ml G418 were treated with trypsin-EDTA (SIGMA) and then released from the plate as cells for which transfection had been confirmed. The number of cells was then counted.

10 days after addition of G418, the entire plate surface was coated with the cells. When the number of the cells was calculated, 9.5×10⁶ cells were found to be present on each 6-well plate. Expression of mutated ERGIC-53 was confirmed by the Western blotting method (Example 8) and the indirect fluorescent antibody method (Example 9) using some of the cells in the following examples.

### EXAMPLE 8

### Confirmation of Expression Using the Western Blotting Method

Whether or not the ERGIC random libraries were expressed precisely in MDCK cells obtained by transfection into the MDCK cells and selection using G418 in the above Example 7 was confirmed using the Western blotting method. In addition, untransfected MDCK cells were used as a control.

Some of cells on day 10 after selection using G418 in Example 7 were transferred into a 24-well plate (FALCON, 3047), and then cultured at 37° C. in 5% CO₂ until the cells filled each well. In addition, MDCK cells used as a control were also inoculated on a 24-well plate (FALCON, 3047) and then cultured similarly.

When the cells filled each well, 50 μl of trypsin-EDTA (SIGMA) was added thereto and the resultant was then cultured at 37° C. in 5% CO₂ for approximately 20 minutes so as to release the cells. 500 μl of a medium was added to the cells released from the plate, centrifugation was carried out at 1000 rpm for 5 minutes, and then the supernatant was discarded. The remained pellet was washed 2 to 3 times with 1×PBS.

The washed pellet of the cells was suspended in 50 μl of 1×PBS. 25 μl of a 2×SDS solubilization buffer (reduction) was added to the suspension, followed by 5 minutes of heat treatment at 100° C. and electrophoresis using 12.5% polyacrylamide gel at 200 V for 45 minutes. In addition, proteins molecular weight marker “Daiichi” III (Daiichi Pure Chemicals) was used as a marker.

By the use of the Western blotting method, the proteins electrophoresed as described above were transferred to PVDF membranes (Immobilon™ Transfer Membranes, Millipore) at 100V for 60 minutes. After transfer, marker portions were cut out, stained with CBB, and then destained. The remaining membranes were immersed in blocking solutions, and then left to stand at 4° C. overnight (the solutions were shaken so that the membranes were uniformly immersed in the blocking solutions).

After blocking, each membrane was put in a Hybri-Bag Soft (COSMO BIO), and then allowed to react with antibodies. As a primary antibody, a solution (4.5 ml) prepared by diluting an anti-FLAG antibody (m2Ab, IBI) using a blocking solution at a concentration of 1 μg/ml was used and then allowed to react at room temperature for 2 hours. Subsequently, the membrane was washed 3 times (5 minutes each) with a washing buffer, and then allowed to react with a secondary antibody. As the secondary antibody, a solution (4.5 ml) prepared by diluting a goat anti-mouse Ig alkaline phosphatase conjugate (BIORAD) at 3000-fold dilution using a blocking solution (5% skim milk was adjusted using TBS-Tween20) was used and allowed to react at room temperature for 30 minutes. Subsequently, the membrane was washed 3 times in total (15 minutes, 5 minutes, and 5 minutes of washing) with a washing buffer. After washing, the membrane was immersed in a chromogenic substrate solution and then left to stand until bands appeared on the membrane. The chromogenic substrate solution was prepared by adding 33 μl of an NBT stock solution (50 mg of NBT was dissolved in 1 ml of 70% DMF (N,N-dimethylformamide) and then stored at −20° C.) and 34 μl of a BCIP stock solution (25 mg of BCIP was dissolved in 1 ml of 50% DMF and then stored at −20° C.) to 5 ml of an alkaline phosphatase substrate buffer containing 100 mM Tris-HCl (pH 9.5), 100 mM NaCl, and 5 mM MgCl₂ in Milli-Q. At a time to stop color development, the membrane was washed in tap water and then air-dried.

The ERGIC random libraries were transfected into MDCK cells, selected with G418 for 10 days, and then subjected to Western blotting, so that expression was confirmed. Compared with the mock transfected MDCK cells, a band that had not been observed in the case of the mock was observed between 66 kDa and 42 kDa molecular weight markers. As indicated in the name ERGIC-53, the molecular weight thereof was 53 kDa and almost in accordance with the position of the band. Hence, it was confirmed that the ERGIC random libraries were expressed in MDCK cells.

### Example 9

### Confirmation of Expression Using Indirect Fluorescent Antibody Method

In the above Example 8, it was confirmed by Western blotting that the MDCK cells into which the ERGIC random libraries had been transfected, and selected with G418 for 10 days, expressed the ERGIC random libraries. Next, through observation using the indirect fluorescent antibody method, the percentage of the transfected cells expressing mutated ERGIC-53 was confirmed. The indirect fluorescent antibody method involves allowing an unlabeled antibody to react with an antigen, binding a fluorescence-labeled secondary antibody to a primary antibody, and then tracing the antigen. A primary antibody functions as an antigen for a secondary antibody, and a fluorescence-labeled immunoglobulin antibody is used as a secondary antibody, whereby the presence or the distribution of an antigen substance of a tissue or cells can be analyzed with good sensitivity. Since the library-composing ERGIC was FLAG-tagged, observation was carried out with a fluorescence microscope using an anti-FLAG antibody (m2Ab, IBI) as a primary antibody and a goat anti-mouse IgG₁-FITC conjugate (Molecular Probes) as a secondary antibody. In addition, untransfected MDCK cells were used as a negative control, and VIP36-FLAG clone 8 (cell line for which constant expression of FLAG-tagged VIP36 had been confirmed) was used as a positive control.

A cover glass (MATSUNAMI) subjected to high-pressure heat sterilization was laid onto a 6-well plate (FALCON, 3046), upon which some (2×10⁵) of the cells after 10 days of selection with G418 in Example 7 were inoculated, and then the resultant was cultured at 37° C. in 5% CO₂ until 90% of the wells were covered with the cells. In addition, untransfected MDCK cells and VIP36-FLAG clone 8 used as controls were also cultured similarly.

On the day of sample preparation, it was confirmed that the cover glass was covered with the cells to some extent. After the media were discarded, the cells were washed once with PBS, to which 1 ml of 4% paraformaldehyde/PBS was added, and then treated at room temperature for 60 minutes, thereby immobilizing them. Subsequently, the cells were washed once with PBS.

The cell membranes were permeabilized by treatment using 0.1% TritonX-100/PBS (1 ml) for 30 minutes at room temperature, followed by 3 times of washing (5 minutes each) with PBS.

Blocking was carried out using a blocking solution (1 ml) at room temperature for 15 minutes, followed by 3 times of washing (5 minutes each) with PBS.

200 μl of the anti-FLAG antibody (m2Ab, IBI) diluted at a concentration of 2 μg/ml using a blocking solution was added onto each cover glass, and then allowed to react at room temperature for 60 minutes, followed by 3 times of washing (10 minutes each) with PBS.

Blocking was carried out again using a blocking solution (1 ml) at room temperature for 15 minutes, followed by 3 times of washing (5 minutes each) with PBS.

200 μl of the anti-mouse IgG₁-FITC (CBL1000F) diluted at a concentration of 2 μg/ml using a blocking solution was added onto each cover glass, light was shielded, and then reaction was carried out at room temperature for 60 minutes, followed by 3 times of washing (10 minutes each) with PBS.

3 μl of DAPI (Molecular Probes) and 3 μl of a slow fade (Molecular Probes) solution A were dropped onto a slide glass (MATSUNAMI). A cover glass was placed on each slide glass so that its cell-adhered face became an undersurface, and then sealed-in using manicure, so as to prepare each sample. Light was shielded for the samples and then they were stored at 4° C.

The prepared samples were observed by a fluorescence microscope (OLYMPUS, BX52). First, negative control MDCK cells were observed, and then positive control VIP36-FLAG clone 8 was observed, so as to confirm whether antibody reaction had been carried out well. Next, the MDCK cells into which the ERGIC random libraries had been transfected and which were then caused to express the libraries were observed. Specifically, several positions were randomly selected from both (left and right) sides of the samples, and then the samples were recorded. The total number of the cells and the number of FITC-positive cells filmed on the photograph were counted. The number of the cells observed to show expression was quantified.

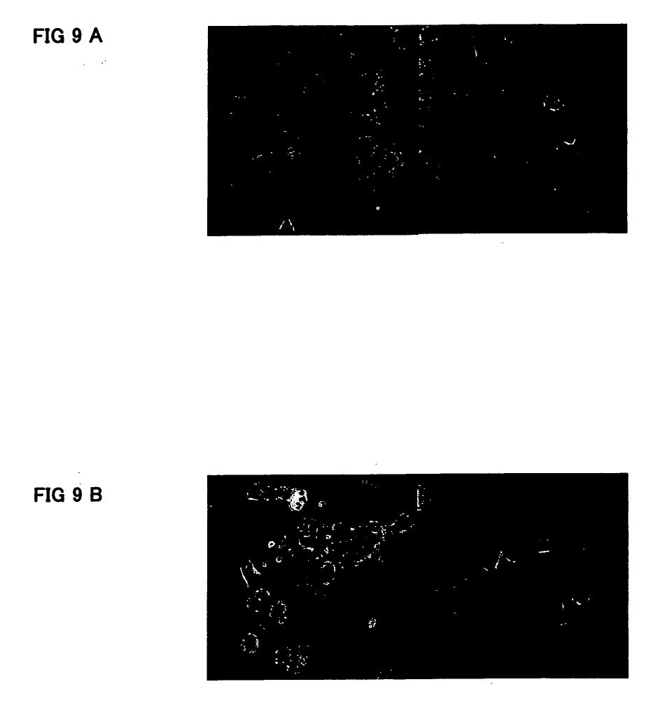

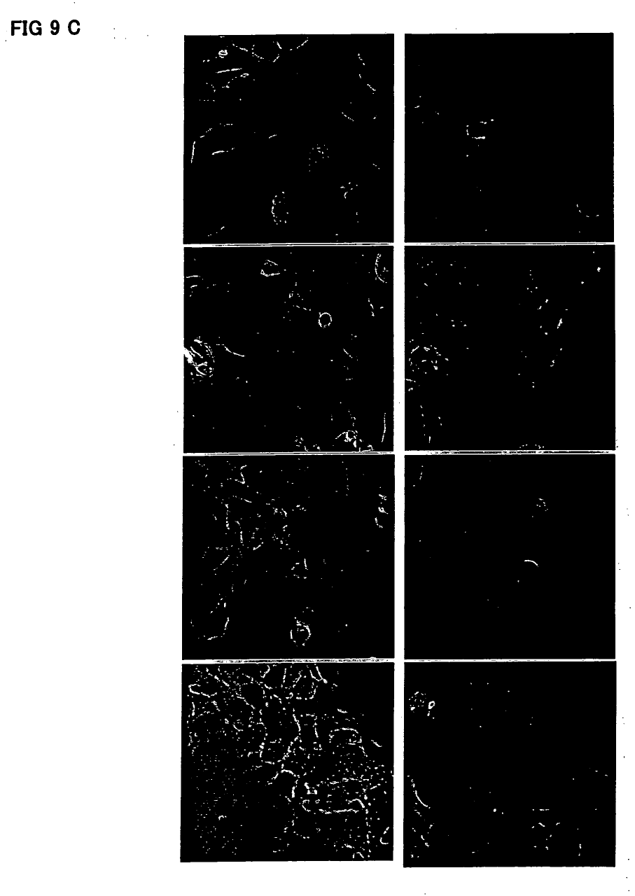

As results, photographs of the samples for a fluorescence microscope are shown in FIG. 9A to C. FIGS. 9A to C show overlay of phase contrast images of the cells (gray) and images of the cells stained with FITC (green). A photograph of the negative control MDCK cells is shown in FIG. 9A and that of the positive control VIP36-FLAG clone 8 is shown in FIG. 9B. In the case of the positive control, each cell emitted very strong green; that is, staining with FITC, the secondary antibody, was observed.

FIG. 9C shows MDCK cells observed after transfection of the ERGIC random libraries and 10 days of selection with 1.5 mg/ml G418. In this figure, 1 or 2 cells per photograph can be seen to have emitted very strong green, indicating strong staining with FITC, the secondary antibody. The total number of the cells filmed on 8 photographs was 387, of which the number of cells strongly stained with FITC was 12. Among samples observed in this time, specifically, it can be said that 12 cells out of 387 cells (3.1%) were cells expressing the libraries strongly.

### EXAMPLE 10

### Screening by MACS Based on Sugar-Binding Specificity of Plant Lectin and FACS Analysis for Cell Expressing ERGIC Random Library

In this example, the sugar-binding specificities of plant lectins were focused on, and cells expressing the ERGIC random libraries were screened by MACS. Specifically, the cells expressing the ERGIC random libraries were primarily labeled with several types of biotinylated Leguminosae lectins, magnetically labeled with streptavidin microbeads, and then screened using a magnetic cell sorting system (Magnetic Cell Sorting and Separation of Biomolecules, hereinafter abbreviated as MACS). Subsequently, after FACS selection, cells were analyzed by FACS.

(1) MACS Screening for Cell Expressing ERGIC Random Library

As biotinylated lectins, Biotin-Lectin sets I and II (Honen Corporation) were used. ConA, BPA, ABA, WGA, SBA, SSA, DBA, DSA, RCA120, LCA, PNA, PHA-L₄, PHA-E₄, Lotus, UEA-I, and MAM (1 mg/ml each) were each diluted with PBS, adjusted to 10 μg/ml, and then sterilized with a 0.45 μm filter (MILLEXOR-HV).

2 days before MACS screening, 4×10⁵ cells were inoculated on sixteen 10-cm dishes, and then cultured at 37° C. in 5% CO₂.

On the day of MACS screening, the cells were washed twice with PBS, to which 5 ml of PBS-0.5 mM EDTA was added, and then the cells were incubated at 37° C. in 5% CO₂, so as to harvest the cells. Centrifugation was carried out at 190×g for 5 minutes, and then the supernatant was discarded.

Each type of lectin (10 μg/ml, 2 ml) was added to the cells for reaction to occur on ice for 30 minutes, centrifugation was carried out for 10 minutes at 30×g, and then the supernatant was removed. After washing with 300 μl of a labeling buffer (prepared using PBS (pH 7.2) and 2 mM EDTA with Milli-Q, followed by filter sterilization), centrifugation was carried out at 300×g for 10 minutes, and then the supernatant was removed.

The cells were suspended in 90 μl of a labeling buffer, to which 10 μl of streptavidin MicroBeads (Miltenyi Biotec GmbH) was added, followed by 15 minutes of reaction at 6° C. to 12° C. After washing with 500 μl of a separation buffer (prepared using PBS (pH 7.2), 2 mM EDTA, and 0.5% BSA with Milli-Q water, followed by filter sterilization), centrifugation was carried out at 300×g for 10 minutes, the supernatant was removed, and then suspension was carried out with 500 μl of a separation buffer.

A MS separation column (Miltenyi Biotec GmBH) was set together with a Mini MACS (Miltenyi Biotec GmbH) magnet, and then 500 μl of a separation buffer was poured into the column. Subsequently, the above cell suspension was applied to the MS separation column, and then the cells eluting out from the column were collected as negative cells (flow-through). 500 μl of a separation buffer was applied 3 times to the MS separation column, and then the cells eluting out from the column were also collected as negative cells (flow-through).

The column was removed from the magnet, and then 1 ml of a separation buffer was applied to the column. The column content was pushed out using the plunger of a syringe and then the cells eluting out from the column were collected as positive cells.

After the obtained positive and negative cells (flow-through) were centrifuged at 300×g for 10 minutes, the supernatant was removed. The pellet was suspended in an appropriate amount of a medium (which had already been supplemented with 1.0 mg/ml G418). The suspension was inoculated on plates with an appropriate size (a 6 cm dish or a 10 cm dish), and then the cells were cultured again.

This experiment was performed with intent to screen for cells based on the types of carbohydrate moieties (sugar chains) added to the membrane proteins on the cell surfaces utilizing the sugar-binding specificities of lectins. The cells labeled with biotinylated lectins and applied to MACS were all divided into negative cells (flow-through) and positive cells. Since it was thought that the cells immediately after MACS had been severely damaged, the number of the cells was counted on the day after MACS screening, and then the ratio at which the cells were divided was confirmed. The results are shown in the following Table 4. However, both negative (flow-through) and positive cells labeled with biotin labeled RCA120 and then subjected to MACS died.

TABLE 4

| Lectin | Negative | |
|---|---|---|
| name | (flow-through) | Positive |
| ConA | 80% | 20% |
| DBA | 88% | 12% |
| LCA | 90% | 10% |
| PHA-E₄ | 85% | 15% |
| PNA | 95% | 5% |
| UEA-I | 80% | 20% |
| WGA | 86% | 14% |
| ABA | 75% | 25% |
| DSA | 70% | 30% |
| Lotus | 93% | 7% |
| MAM | 20% | 80% |
| PHA-L₄ | 82% | 18% |
| SBA | 85% | 15% |
| SSA | 60% | 40% |
| BPA | 90% | 10% |

As is understood from Table 4 above, most cells selected by MACS in this example were collected as flow-through. Differences depending on the lectins used were not clearly observed.

Among the cell fractions obtained in this time, negative cells flow-through) were designated (−), cells that had passed through the column after being recognized as positive cells and selected by MACS were designated ConA (−), and cells finally collected as positive cells were designated ConA (+). Regarding other lectins, similar notation was employed. A total of 30 types of these cells were subjected to FACS analysis in the following experiments.

(2) FACS Analysis

30 types of cells that had been selected by MACS in (1) above and then grown successfully were analyzed by FACS. Specifically, the cells were allowed to react with 15 types of biotinylated lectins as primary antibodies and FITC-labeled streptavidin (PIERCE) as a secondary antibody, and then changes in fluorescence intensity were observed using FACS. In addition, untransfected MDCK cells were used as a control.

On the day before FACS, 2×10⁵ cells to be analyzed were inoculated on a 6-cm plate. On the day of FACS, each type of cell was washed twice with PBS, to which 2.5 ml of PBS-0.5 mM EDTA was added, and then the cells were cultured at 37° C. in 5% CO₂, so as to release the cells. When the cells were released, 2.5 ml of a FACS buffer (prepared by dissolving 9.8 g of HBSS (NISSUI PHARMACEUTICAL CO., LTD.) in Milli-Q, and then to which 0.35 g of NaHCO₃, 10 ml of 10% NaN₃, and 5 ml of 20% BSA/NaN₃ solution were added, followed by adjustment of the solution to 1 litter) was added. The solution was transferred into a 15-mI tube, centrifugation was carried out at 1000 rpm for 10 minutes, and then the supernatant was discarded. Furthermore, the cells were washed once with 5 ml of a FACS buffer, the number of the cells was determined, centrifugation was carried out at 190×g for 10 minutes, and then the supernatant was discarded.

The cells were diluted with a FACS buffer to a concentration of 8×10⁶/ml (4×10⁵/50 μl). The diluted solution was transferred in amount of 50 μl into a 96-well U-bottom plate (3077, FALCON). 50 μl of 10 μg/ml biotinylated lectin was added as a primary antibody to each well. The solution was gently stirred, and then allowed to react on ice for 30 minutes.

100 μl of a FACS buffer was added, the solution was centrifuged at 580×g for 3 minutes, and then the supernatant was discarded. The pellet was applied to Vortex mixer to disassemble it. A similar washing operation was further repeated twice (in addition, washing was carried out by adding 200 μl of a FACS buffer at each time).

50 μl of streptavidin FITC (diluted with a FACS buffer to 20 μg/ml) was added as a secondary antibody. The solution was lightly stirred, light was shielded, and then the solution was allowed to react on ice for 20 to 30 minutes.

150 μl of a FACS buffer was added, the solution was centrifuged at 580×g for 3 minutes, and then the supernatant was discarded. A similar washing operation was repeated once more.

200 μl of a FACS buffer was added to each well. After pipetting, the solution was transferred into a FACS tube. 100 μl of 3 μg/ml propidium iodide (SIGMA) was added, and then dead cells were stained. The cells were analyzed by CellQuest (Becton-Dickinson Immunocytometory Systems) using FACSCalibur. Viable cells were selected based on forward scattered light, lateral scattered light, and staining with propidium iodide (SIGMA). Information on 10,000 viable cells was collected and analyzed.

In the ERGIC random library, random mutations had been introduced into amino acids corresponding to the putative carbohydrate-binding domain of ERGIC-53. ERGIC-53 is a molecule that exists in transport vesicles moving between the endoplasmic reticulum (ER) and the cis Golgi and selectively transports (because of the sugar-binding specificity of carbohydrate recognition domain, it binds to mannose in a Ca²⁺-dependent manner) glycoproteins. Hence, it is considered that through introduction of random mutations into a carbohydrate recognition domain or a carbohydrate-binding domain, sugar-binding specificities differing from the original specificity of ERGIC-53 are generated in the ERGIC random library, and mutated ERGIC-53 will bind to glycoproteins having various carbohydrate moieties, so that the carbohydrate moiety-types of glycoproteins transported onto the cell membrane surface will be varied. In MACS carried out in Example 10, cells were sorted based on the sugar-binding specificities (see the following Table 5) of various biotinylated lectins used herein, and then collected as separate fractions. Generally, when a lectin binds to a carbohydrate moiety, it recognizes a size of around that of a trisaccharide, and identifies the three-dimensional structure. Analysis using FACS reveals what type of carbohydrate moiety is present on the cell surface at what ratio by staining with biotinylated lectins.

TABLE 5

| Lectin | | |
|---|---|---|
| name | Origin | Specificity |
| ConA | Canavalia ensiformis | α-D-Man |
| | Jack bean | α-D-Glc |
| DBA | Dolichos biflorus | D-GalNAc |
| LCA | Lens culinaris | α-D-Glc |
| | | α-D-Man |
| PHA-E₄ | Phaseolus Vulgaris | D-GalNAc |
| PNA | Peanut (Arachis hypogaea) | D-Gal |
| | | Galβ1-3 GalNAc |
| UEA-I | Ulex europaeus | β-L-Fuc |
| WGA | Wheat Germ | D-GlcNAc |
| ABA | Agaricus bisporus | β-D-Gal |
| DSA | Datura stramonium | β-D-GlcNAc |
| | (foreign species) | Galβ1-4GalNAc |
| Lotus | Lotus Itetragonolous | α-L-Fuc |
| MAM | Maackia amurensis | SAα2-3Gal |
| PHA-L₄ | Phaseolus Vulgaris | D-GalNAc |
| SBA | Soybean | D-GalNAc |
| SSA | Sambucus sieboldiana | SAα2-6Gal/GalNAc |
| BPA | Bauhinia purpurea | D-GalNAc > D-Gal |

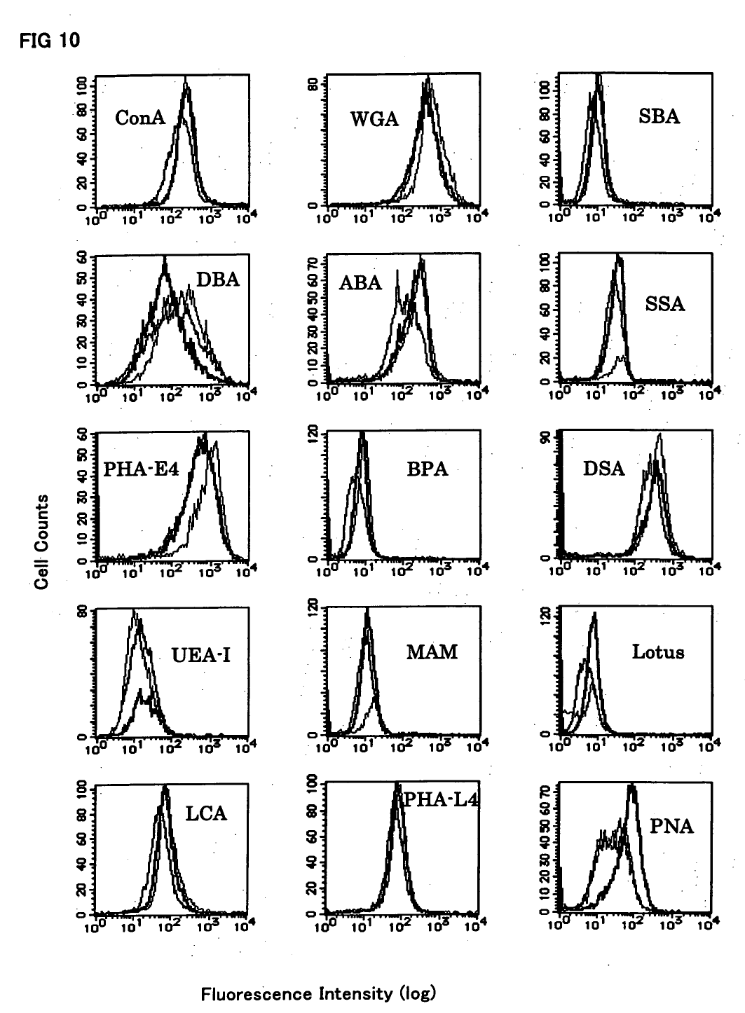

The results of FACS analysis are shown in FIG. 10. In FIG. 10, cells following MACS screening and control cells (wild type MDCK cells) were compared, and then the difference in average fluorescence intensity was observed. In FIG. 10, a black line indicates the control MDCK cells, a red line indicates the (−) fraction following MACS, and a green line indicates the (+) fraction. Those showing average fluorescence intensity of the (+) fraction and that of the (−) fraction higher than that of the control were only the cells obtained by primary labeling with PNA and ABA, followed by MACS screening. Specifically, it can be said that among ERGIC random library-expressing cells, cells presenting carbohydrate moieties specifically bound to PNA lectin on the cell surfaces were selected by MACS.

SBA, DBA, PHA-E₄, PHA-L₄, and BPA are all lectins specifically binding to D-GalNAc. As shown in the FACS result in FIG. 10, in the case of all lectins, almost no differences were observed in average fluorescence intensity between the control and (+) and (−) fractions.

### EXAMPLE 11

### Analysis of Cell Specific to PNA Lectin

In this example, through further screening by MACS of the PNA (+) cells obtained in Example 10, we tried to enrich cells presenting more carbohydrate moieties specifically bound to PNA lectin on the cell surfaces. Furthermore, analysis was carried out by FACS and lectin staining using the Western blotting method.

(1) MACS Separation and FACS Analysis of PNA(+)

Screening of the PNA(+) cells obtained in Example 10 was carried out by MACS, and then the cells were analyzed by FACS. In addition, as a control for FACS, untransfected MDCK cells were used.

(1-1) Screening for PNA (+) by MACS

4×10⁵ cells (PNA(+)) were inoculated on a 10 cm-dish (FALCON, 3003) and then cultured at 37° C. in 5% CO₂ for 2 days.

The cells were washed twice with PBS, to which 5 ml of PBS-0.5 mM EDTA was then added, so as to harvest the cells. Centrifugation was carried out at 190×g for 5 minutes, and then the supernatant was discarded.

Biotinylated PNA lectin (10 μg/ml, 2 ml) was added to the cells for binding to occur on ice for 30 minutes. Centrifugation was carried out at 300×g for 10 minutes, and then the supernatant was removed. After washing with 300 μl of a labeling buffer, centrifugation was carried out at 300×g for 10 minutes, and then the supernatant was removed.

The cells were suspended in 90 μl of a labeling buffer, to which 10 μl of streptavidin MicroBeads (Miltenyi Biotec GmbH) was then added, followed by 15 minutes of reaction at 6° C. to 12° C. After washing with 500 μl of a separation buffer, 10 minutes of centrifugation at 300×g, and then removal of the supernatant, the product was suspended in 500 μl of a separation buffer.

A MS separation column (Miltenyi Biotec GmBH) was set together with a MiniMACS (Miltenyi Biotec GmbH) magnet, and then 500 μl of a separation buffer was applied to the column. Subsequently, the above cell suspension was applied to the MS separation column, and then the cells eluting out from the column were collected as negative cells (flow-through). 500 μl of a separation buffer was applied 3 times to the MS separation column, and then the cells eluting out from the column were also collected as negative cells (flow-through).

The column was removed from the magnet, and then 1 ml of a separation buffer was applied to the column. The column content was pushed out using the plunger of a syringe and then the cells eluting out from the column were collected as positive cells.

After the obtained positive and negative cells (flow-through) were centrifuged at 300×g for 10 minutes, the supernatant was removed. The pellet was suspended in an appropriate amount of a medium (that had already been supplemented with 1.0 mg/ml G418). The suspension was inoculated on dishes with an appropriate size (a 6 cm dish or a 10 cm dish), and then the cells were cultured again.

As a result, 65% of the collected cells were found to be negative cells (flow-through) and 35% of the collected cells were found to be positive cells. Hereinafter, negative cells collected by applying PNA(+) to MACS are referred to as PNA2(−), and positive cells collected by the same are referred to as PNA2(+). At the 1^{st} round of MACS (Example 10), 95% of the cells were PNA(−) and 5% of the same were PNA (+). At the 2^{nd} round of MACS, the percentage of the cells collected as the (+) fraction increased.

(1-2) FACS Analysis

On the day before FACS, 2×10⁵ cells to be analyzed were plated on a 6-cm dish (FALCON, 3002). On the day of FACS, each type of cell was washed twice with PBS, to which 5 ml of PBS-0.5 mM EDTA was then added, and then the cells were cultured at 37° C. in 5% CO₂, so as to release the cells. When the cells were released, 5 ml of a FACS buffer was added. The solution was transferred into a 15-ml tube, centrifugation was carried out at 190×g for 10 minutes, and then the supernatant was discarded. Furthermore, the cells were washed once with 5 ml of a FACS buffer, the number of the cells was determined, centrifugation was carried out at 190×g for 10 minutes, and then the supernatant was discarded.

The cells were diluted with a FACS buffer to a concentration of 8×10⁶/ml (4×10⁵/50 μl). The diluted solution was transferred in amount of 50 μl into a 96-well U-bottom plate (3077, FALCON). 50 μl of 10 μg/ml biotinylated PNA lectin was added as a primary antibody to each well. The solution was gently stirred, and then allowed to react on ice for 30 minutes.

100 μl of a FACS buffer was added, the solution was centrifuged at 580×g for 3 minutes, and then the supernatant was discarded. The pellet was applied to Vortex mixer to disassemble it. A similar washing operation was further repeated twice.

50 μl of streptavidin FITC (diluted with a FACS buffer to 20 μg/ml) was added as a secondary antibody. The solution was slightly stirred, light was shielded, and then the solution was allowed to react on ice for 20 to 30 minutes.

150 μl of a FACS buffer was added, the solution was centrifuged at 580×g for 3 minutes, and then the supernatant was discarded. The pellet was applied to Vortex mixer to disassemble it. Similar washing operation was repeated once more (in addition, washing was carried out by adding 200 μl of a FACS buffer at each time).

200 μl of a FACS buffer was added to each well. After pipetting, the solution was transferred into a FACS tube. 100 μl of 3 μg/ml propidium iodide (SIGMA) was added, and then dead cells were stained. The cells were analyzed by CellQuest (Becton-Dickinson Immunocytometory Systems) using FACSCalibur. Viable cells were selected based on forward scattered light, lateral scattered light, and staining with propidium iodide (SIGMA). Information on 10,000 viable cells was collected and analyzed.

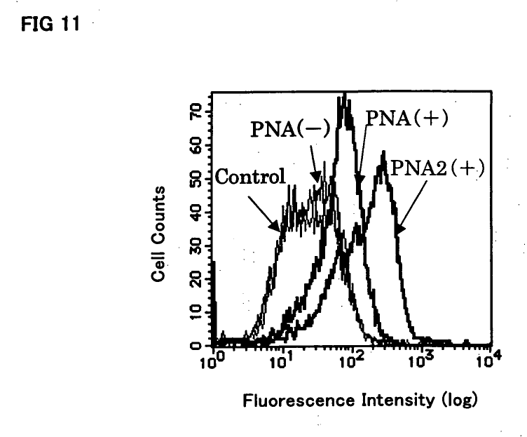

The results are shown in FIG. 11. PNA(−), PNA(+), and PNA2(+) were compared. As a control, untransfected MDCK cells were used. As shown in FIG. 11, the average fluorescence intensity of PNA2(+) was higher than that of PNA(+), suggesting that cells presenting more carbohydrate moieties (sugar chains) specifically bound to PNA on the cell surfaces were collected in PNA2(+).

(2) Lectin Staining Using the Western Blotting Method

Based on the result of (1) above, it was considered that PNA2(+) was cells group having more carbohydrate moieties specifically bound to the PNA lectin on thee cell surfaces compared with the case of PNA(+) or PNA(−). Hence, in this example, lectin staining was carried out using the PNA lectin by the Western blotting method, and then differences in the degree of staining among PNA(−), PNA(+), and PNA2(+) were observed. Furthermore, through the use of 4 types of lectins (ConA, MAM, PHA-E₄, and PHA-L₄), lectin staining was carried out by the Western blotting method using lectins having different sugar-binding specificities from that of PNA, followed by comparison. In addition, as a control, untransfected MDCK cells were used.

Cells were inoculated on a 6-well plate (FALCON, 3046), and then cultured at 37° C. in 5% CO₂ until the cells filled about 90% of each well.

When the cells had sufficiently grown, 500 μl of cells lysis buffer (10 mM EDTA, 0.2% Triton X-100, 1 mM PMSF, and 1 μg/ml leupeptin were adjusted using PBS) was added to the cells, and then the solution was placed on ice for 1 to 1.5 hours so as to lyse the cells.

After treatment with the cell lysis buffer, the cell lysates were subjected to protein quantitative determination using a BCProteins assay kit (PIERCE). The cell lysates were diluted using PBS at a concentration that was the same as the lowest concentration. 10 μl of each cell lysate diluted at the lowest protein concentration was transferred into a 1.5 ml-tube, to which 2 μl of a 6×SDS solubilization buffer (reduction) was then added, followed by 5 minutes of heat treatment at 100° C. Subsequently, electrophoresis was carried out at 200 V for 45 minutes using 12.5% acrylamide gel.

By the use of the Western blotting method, the above-electrophoresed proteins were transferred to PVDF membranes (Immobilon™ Transfer Membranes, Millipore) at 100 V for 60 minutes. After transfer, marker portions were cut out, stained with CBB, and then destained. The remaining membranes were immersed in blocking solutions and left to stand at 4° C. overnight (the solutions were shaken so that the membranes were uniformly immersed in the antibody-containing blocking solutions).

After blocking, each membrane was put in a Hybri-Bag Soft (COSMO BIO), and then allowed to react with antibodies. As a primary antibody, a solution (4.5 ml) prepared by diluting a biotinylated lectin with a blocking solution to a concentration of 1 μg/ml was used, and allowed to react at room temperature for 2 hours. Subsequently,.the membrane was washed 3 times (5 minutes each) with a washing buffer, and then allowed to react with a secondary antibody. As the secondary antibody, a solution (4.5 ml) prepared by diluting streptavidin alkaline phosphatase with a blocking solution to a concentration of 1 μg/ml was used and allowed to react at room temperature for 30 minutes. Subsequently, the membrane was washed 3 times in total (15 minutes, 5 minutes, and 5 minutes of washing) with a washing buffer. After washing, the membrane was immersed in a chromogenic substrate solution and then left to stand until bands appeared on the membrane. At a time to stop color development, the membrane was washed in tap water and then air-dried.

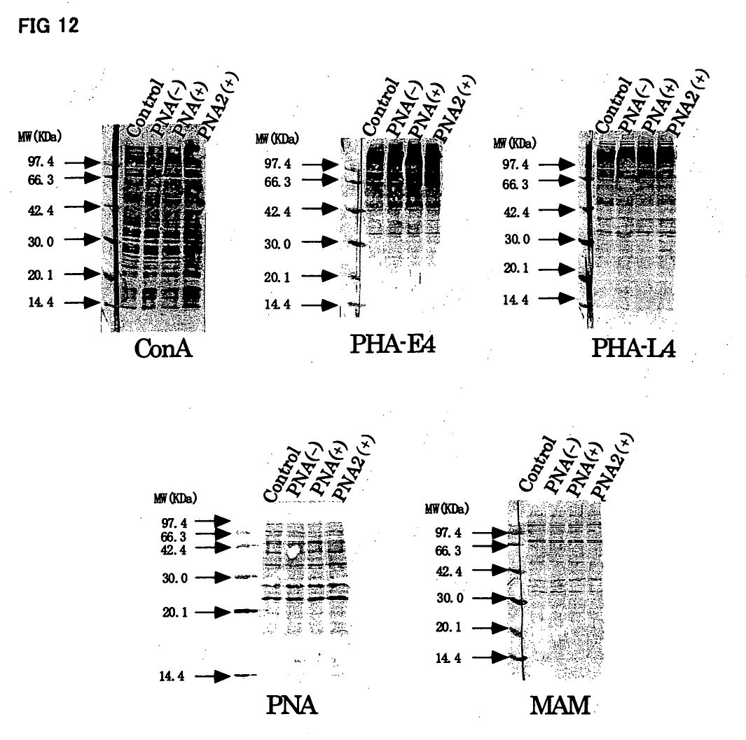

The results are shown in FIG. 12. In the results of Western blotting using PNA, band thickness was observed in the descending order of PNA2(+)>PNA(+)>PNA(−)>control. However, no significant differences were observed, suggesting that the specificities to PNA were at the same level for all the cell fractions analyzed in this time. Furthermore, in the result of Western blotting using ConA, PHA-E₄, and PHA-L₄, no large differences were observed between the control and PNA(−), PNA(+), or PNA2(+) in terms of the degree of staining. In the results of Western blotting using MAM, a slight difference in band thickness was observed in the descending order of the control>PNA(−)>PNA(+)>PNA2(+).

Next, when the degree of staining with each lectin was compared, thickness as a result of staining was observed in the descending order of ConA>PHA-E₄, PHA-L₄>PNA>MAM in every case regarding the control, PNA(−), PNA(+), and PNA2(+).

Here, the sugar-binding specificity of each lectin used in Western blotting in this experiment is explained. ConA is a lectin showing affinity for α-D-Man and α-D-Glc and binds to biantennary complex-type and high mannose-type, and hybrid-type sugar chains. Furthermore, both PHA-L₄ and PHA-E₄ show affinity for D-GlcNAc and bind to complex-type carbohydrate moiety among asparagine-binding-type carbohydrate moieties. PNA has affinity for D-Gal and is a lectin specific to a Ser/Thr-type carbohydrate moiety so that it particularly strongly binds to Galβ1-3GalNAc. Moreover, MAM has specificity to SAα2-3Gal and is a lectin specific to sialic acid so that it binds strongly to SAα2-3Galβ1-3GalNAc. In terms of the biosynthetic process of oligosaccharides, the high mannose-type oligosaccharides are added in the cis Golgi (ER), the complex-type oligosaccharides are processed in the medial and trans Golgi, and the Ser/Thr linked oligosaccharides are added in the trans Golgi. Carbohydrate moieties having sialic acids are added in the final processing step in the trans Golgi. There may be correlation between these facts and the results of Western blotting in this experiment wherein the band thickness was detected in the descending order of ConA>PHA-E₄, PHA-L₄>PNA>MAM. Specifically, Western blotting using lectins specific to carbohydrate moieties to be added at earlier stages in oligosaccharide processing resulted in thicker staining. This suggests the possibility that most of cellular glycoproteins were being biosynthesized and that differences among glycoproteins expressed on the cell surfaces were relatively small.

(3) Glycosylation in the Trans Golgi

As a result of Western blotting carried out in (2) above, in the case of Western blotting using MAM and PNA, only slight differences were observed in intensity of band among each sample of the control, PNA(−), PNA(+), and PNA2(+). Hence, in this example, intensity of band resulting from Western blotting were quantitatively determined. It was considered that since intra- and extrcellsular proteins are lysed together and analyzed in Western blotting, intracellular immature glycoproteins were predominant, so that it is impossible to clearly observe differences between such intracellular glycoproteins and membrane glycoproteins having binding specificity to PNA. Thus, analysis by flow cytometry was also carried out.

(3-1) Analysis Using Image Analyzer

The membranes obtained by Western blotting in (2) above were quantitatively determined using an image analyzer (LAS-l000, FUJI PHOTO FILM), and then intensity of band was compared.

Analysis using the image analyzer involves finding the average value per unit area of a band to be quantitatively determined and comparing average values. The results are shown in the following Table 6.

TABLE 6

| | PNA | MAM |
|---|---|---|
| Control | 4554000 | 4792000 |
| PNA(−) | 4618000 | 4776000 |
| PNA(+) | 4638000 | 4750000 |
| PNA2(+) | 4747000 | 4714000 |

As shown in Table 6 above, in the case of Western blotting using PNA, intensity of band was observed in the ascending order of the control<PNA(−)<PNA(+)<PNA2(+) and in the case of Western blotting using MAM, the intensity of band was observed in the descending order of the control>PNA(−)>PNA(+)>PNA2(+). However, in each case, the difference was as subtle as approximately 1%.

(3-2) FACS Analysis

Next, analysis focusing on membrane glycoproteins on cell surfaces was carried out using FACS. On the day before FACS, 2×10⁵ cells to be analyzed (control MDCK cells, PNA(−), PNA(+), and PNA2(+)) were inoculated on a 6-cm dish (FALCON, 3002). On the day of FACS, each type of cell was washed twice with PBS, to which 5 ml of PBS-0.5 mM EDTA was then added, and then the cells were incubated at 37° C. in 5% CO₂, so as to harvest the cells. When the cells were released, 5 ml of a FACS buffer was added. The solution was transferred into a 15-ml tube, centrifugation was carried out at 190×g for 10 minutes, and then the supernatant was discarded. Furthermore, the cells were washed once with 5 ml of a FACS buffer, the number of the cells was determined, centrifugation was carried out at 190x g for 10 minutes, and then the supernatant was discarded.

The cells were diluted with a FACS buffer to a concentration of 8×10⁶/ml (4×10⁵/50 μl). The diluted solution was transferred in amount of 50 μl into a 96-well U-bottom plate (3077, FALCON). 50 μl of 10 μg/ml biotinylated PNA lectin or MAM lectin was added as a primary antibody to each well. The solution was gently stirred, and then allowed to react on ice for 30 minutes.

100 μl of a FACS buffer was added, the solution was centrifuged at 580×g for 3 minutes, and then the supernatant was discarded. After a Kim towel was held to the pellet to remove water, the pellet was applied to Vortex mixer to disassemble it. A similar washing operation was further repeated twice.

50 μl of streptavidin FITC (diluted with a FACS buffer to 20 μg/ml) was added as a secondary antibody. The solution was lightly stirred, light was shielded, and then the solution was allowed to react on ice for 20 to 30 minutes.

150 μl of a FACS buffer was added, the solution was centrifuged at 580×g for 3 minutes, and then the supernatant was discarded. After a Kim towel was held to the pellet to remove water, the pellet was applied to Vortex mixer to disassemble it. A similar washing operation was repeated once more.

200 μl of a FACS buffer was added to each well. After pipetting, the solution was transferred into a FACS tube. 100 μl of 3 μg/ml propidium iodide (SIGMA) was added, and then dead cells were stained. The cells were analyzed by CellQuest (Becton-Dickinson Immunocytometory Systems) using FACSCalibur. Viable cells were selected based on forward scattered light, lateral scattered light, and staining with propidiuin iodide (SIGMA). Information on 20,000 viable cells was collected and analyzed.

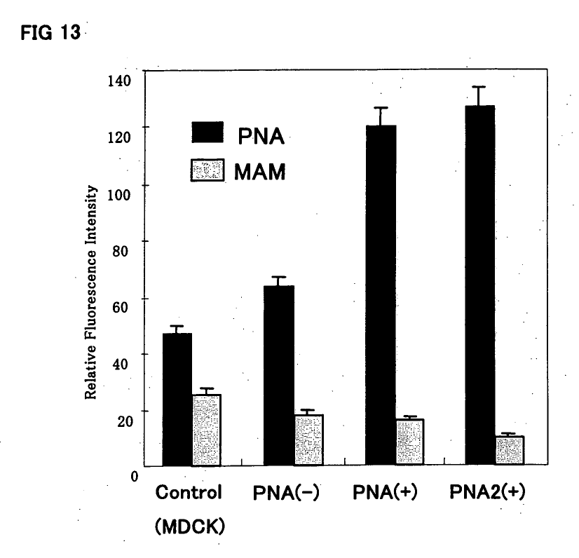

By the use of biotinylated lectin (MAM or PNA) as a primary antibody and streptavidin FITC as a secondary antibody, fluorescence intensity was measured for 4 cell fractions (control MDCK wild-type cells, PNA(−), PNA(+), and PNA2(+)) and the average values thereof were compared. The results are shown in FIG. 13. In FIG. 13, in the results of FACS using PNA as a primary antibody, the value of the average fluorescence intensity was observed in the ascending order of the control<PNA(−)<PNA(+)<PNA2(+). In the meantime, in the results of FACS using MAM as a primary antibody, the value of the average fluorescence intensity was observed in the descending order of the control>PNA(−)>PNA(+)>PNA2(+). Specifically, the results of FACS using MAM and the results of FACS using PNA showed an inverse correlation. Furthermore, the results were in accordance with the results of analysis of the degree of staining in Western blotting using an image analyzer. Here, in terms of oligosaccharide processing, the results are considered. To Ser/Thr linked oligosaccharides, carbohydrate moieties are added in the order of GalNAc-Ser/Thr→Galβ1-3GalNAc-Ser/Thr→SAα2-3Galβ1-3GalNAc-Ser/Thr. PNA specifically recognizes and binds to Galβ1-3GalNAc-Ser/Thr, but does not recognize at all when SA (sialic acid) is added thereto. On the other hand, MAM is a lectin that specifically recognizes and binds to SAα2-3Galβ1-3GalNAc-Ser/Thr, but does not recognize any sugar chains at a stage where a sialic acid has not yet been added thereto. When the above facts and the results shown in FIG. 13 are taken together, it is considered that cell fractions presenting more Galβ1-3GalNAc-Ser/Thr on the cell surfaces thereof were collected in PNA2(+).

In Examples 5 to 11 above, among 9 amino acids (DTFDNDGKK) corresponding to the putative carbohydrate-binding domain of ERGIC-53, random mutations were introduced into 7 amino acids (DXXXNXXXX; X denotes any amino acid) and then introduced into pRc-CMV2, the vector. Thus, ERGIC-53 random libraries were constructed, the libraries were transfected into MDCK cells, and then the cells were forced to express recombinant ERGIC-53. In this specification, the cells are referred to as “cells expressing ERGIC random libraries.” After transfection, selection using G418 was carried out, and in order to screen for cells expressing glycoproteins having various carbohydrate moieties on the cell surfaces thereof, the cells were primarily labeled using various biotinylated lectins, magnetically labeled with streptavidin MicroBeads, and then screening was carried out using the magnetic cell sorting system (MACS). As a result, MDCK cells reacting with several types of lectins could be separated; that is, cells having specific lectin-binding activity (that is able to specifically recognize carbohydrate moieties) could be obtained.

### EXAMPLE 12

### Screening of Mutated-VIP36-Transfected Cell Using FACS and Enrichment of Cell Expressing a Glycoprotein With a Modified Carbohydrate Moiety

(1) Construction of Mutated VIP36-Expressing CHO Cell Library

CHO (Chinese hamster ovary cells) cells (provided by the Cell Resource Center for Biomedical Research, Tohoku University) were cultured in RPMI1640 supplemented with 10% fetal bovine serum (FBS) in the presence of 5% CO₂. CHO cells cultured to be confluent were collected by trypsin/EDTA, diluted 10-fold, and then further cultured overnight. 12 μg of plasmids (linear) of the mutated VIP36 gene library constructed in Example 1 was used for transfection into 2.4×10⁶ CHO cells according to a standard method using Lipofectamine 2000 (GIBCO). After the cells were cultured for 48 hours in RPMI1640 containing 10% FBS in the presence of 5% CO₂, the cells were cultured in the same culture medium containing 1.2 mg/ml G418 (CalbioChem) for 10 to 14 days. Thus, only cell lines constitutively expressing mutated VIP36 were selected.

(2) Screening for Altered-VIP36-Expressing Cell by Flow Cytometry

The altered VIP36-expressing CHO cells prepared in (1) above were diluted to be 10% confluent in a 10 cm-dish, and then cultured. After 48 hours, the cells were released and collected using a phosphate buffer (pH 7.4) (EDTA/PBS) containing 0.5 mM EDTA and 150 mM NaCl, and then suspended in 0.5 ml of a FACS buffer. Next, biotinylated PNA lectin was added as a primary antibody at 5 μg/ml, and then left to stand on ice for 30 minutes. After excessive biotinylated PNA lectin was washed off using a FACS buffer (Becton Dickinson), FITC-labeled streptavidin was added as a secondary antibody at a concentration of 10 μg/ml, and then similarly allowed to react on ice for 20 minutes. After washing with a FACS buffer, an aggregated cell mass was removed by filtration through nylon mesh. The cells were suspended in a FACS buffer at a concentration of 10⁵ cells/ml. The suspension was stored on ice until sorting.

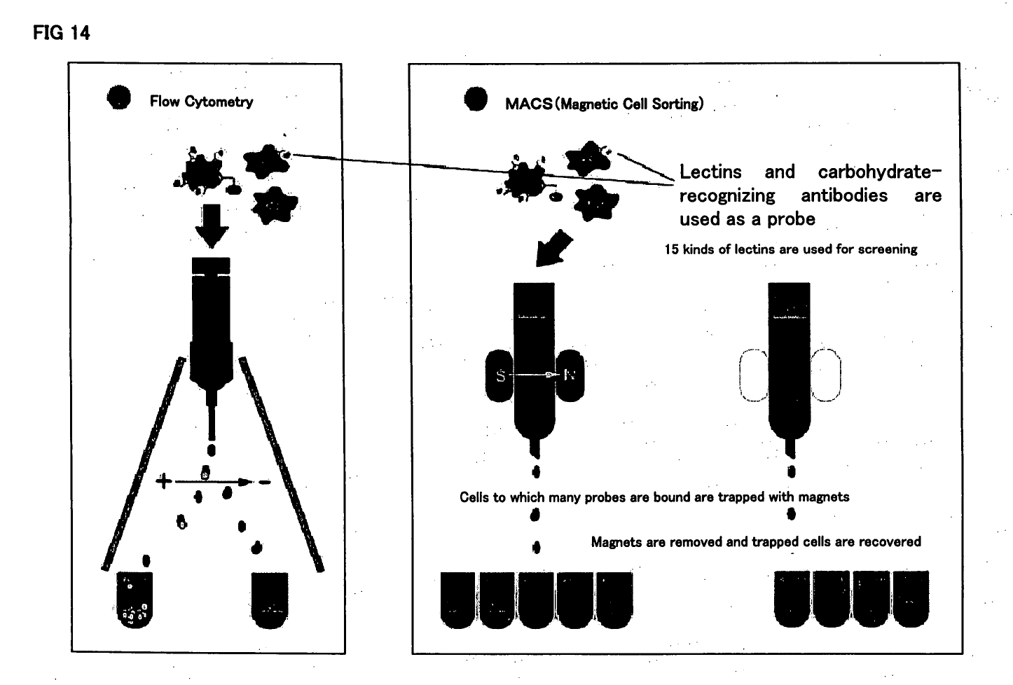

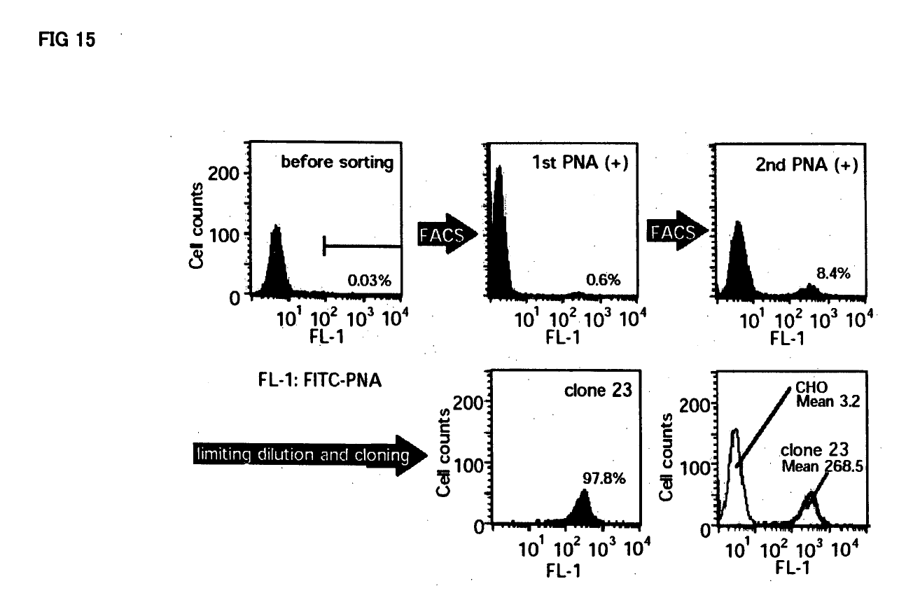

For cell screening, flow cytometry was used (FIG. 14) instead of using the method utilizing MACS as described in Example 3. Flow cytometry was carried out using FACS Vantage (produced by Becton Dickinson). Following optical axis adjustment and channel sterilization, the cells prepared as described above were sorted according to the manuals. A screening window was provided for a PNA lectin-positive cell fraction to be approximately around 0.1%. 1.4×10⁶ cells were sorted, and then 1,187 PNA-lectin-positive cell fractions were obtained. In tubes for collection, lactose, the hapten sugar of PNA, with a concentration of 20 μM, had been previously added to FACS buffer. Positive cells were inoculated on a 24-well dish, and then cultured in RPMI1640 containing 10% FBS in the presence of 5% CO₂. Lactose was used for releasing PNA lectin from PNA lectin-positive cells, because PNA lectin bound to the cells may provide a cytotoxic effect. 2 weeks later, the cells were stained with PNA lectin, and then the proportion of PNA-positive cells was examined (FIG. 15, 1 st PNA(+)).

The second sorting was carried out by a method similar to that of the first sorting by providing a screening window for a PNA lectin-positive cell fraction to be approximately around 0.25% (2,352 cell fractions/6.3×10⁵ cells). After 2 weeks of culture, the cells were stained with PNA lectin, and then the percentage of PNA-positive cells was examined (FIG. 15, 2nd PNA(+)).

(3) Cloning of PNA Lectin-Positive Cells

The PNA-lectin-positive cells collected in the second sorting were suspended in 10 ml of RPMI1640 containing 10% FBS. The suspension was inoculated in a flat-bottomed 96-well plate with 200 μl of the suspension in each of 50 wells, and then cultured. At the time when colonies within the wells could be visually confirmed, wells having a concentration of 1 colony per well were selected and then collected with EDTA/PBS, followed by subculture in a 24-well plate. 19 out of 50 wells had 1 colony per well. At the time when the culture in the 24-well plate became confluent, binding of each cell clone to PNA lectin was examined. Among 19 wells, the cells in 13 wells were strongly stained with PNA, and a plurality of PNA lectin-positive cell clones were obtained. One of these clones, clone 23, is shown in FIG. 15 (Clone 23). The upper row of FIG. 15 shows fluorescence intensity before sorting, that after the 1st sorting, and that after the 2nd sorting. The lower row in FIG. 15 shows fluorescence intensity of clone 23 obtained after the 2nd sorting. As shown in the graph at the lower right in FIG. 15, FITC fluorescence intensity was 3.2 in the case of the CHO cells, the parent line, whereas in the case of clone 23, it was 268.5, which was approximately 100 times greater than the former fluorescence intensity.

(4) Examination of FLAG Tag Expression of PNA Lectin-Positive Cell Clone 12

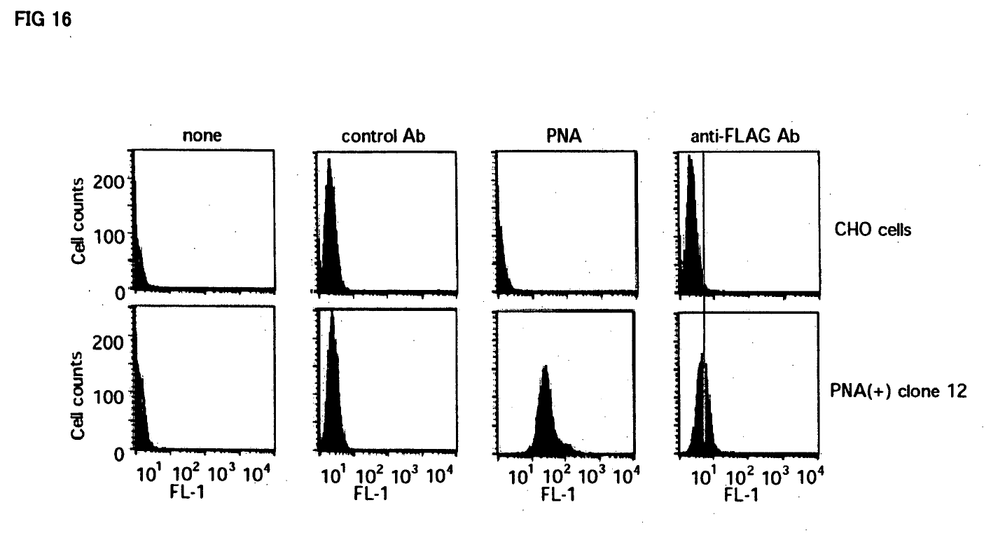

For one of the clones obtained in (3) above (clone 12), whether or not mutated VIP36, the product of the foreign gene, was expressed was examined using anti-FLAG antibody. Upon constructing a mutated VIP36 gene, it was designed such that a FLAG tag sequence was added to the N-terminus. Hence, the expression of altered VIP36 can be examined by staining it with the anti-FLAG antibody. Thus, PNA lectin-positive cell clone 12 cultured to be 70% confluent was harvested with EDTA/PBS and then allowed to react with both the anti-FLAG antibody (3.3 1g/ml) and biotinylated PNA (3.3 μg/ml) simultaneously on ice for 30 minutes. After washing with a FACS buffer, the clone was further allowed to react with FITC-labeled anti-mouse IgG (5 μg/ml) and PE (phycoerythrin)-labeled streptavidin (1 μg/ml) on ice for 20 minutes. Fluorescence intensity of the cells was analyzed using an analyzer (FACSCalibur) (FIG. 16). In FIG. 16, “none” represents the result when nothing corresponding to a primary antibody had been added. “Control Ab” represents the result when an anti-rat IgG antibody known not to bind to CHO cells had been allowed to react as a primary antibody to stain the cells, “PNA” represents the result when the biotinylated PNA lectin had been allowed to react as a primary antibody to stain the cells, and “anti-FLAG Ab” represents the result when the anti-FLAG antibody had been allowed to react as a primary antibody to stain the cells. Although the expression level in clone 12 cells was low, clone 12 cells were clearly FLAG-positive.

(5) Analysis of Mutated VIP36 Gene Expressed on PNA-Positive Clones

mRNA was extracted from various PNA lectin-positive cell clones using a μ MACS system (Daiichi Pure Chemicals). The cells cultured to be 50% to 60% confluent in four 10-cm dishes were collected with trypsin/EDTA, and then suspended in a lysis/binding buffer (Daiichi Pure Chemicals, attached within a kit) so as to disrupt the cells. mRNA was annealed to oligo (dT) microbeads (Daiichi Pure Chemicals), and then applied to a MACS column. The column was further washed with a lysis/binding buffer and a washing buffer (Daiuchi Pure Chemicals, attached within a kit), and then an elution buffer (Daiichi Pure Chemicals, attached within a kit) was added so as to elute mRNA from the column. 6.3 mg of mRNA was obtained from 1.1×10⁷ cells.

1 mg of mRNA was dissolved in 20 μl of Milli-Q water, and then to which 1 μg of oligo(dT) was added. The solution was heated at 70° C. for 10 minutes, and then chilled on ice. 2 μl of 10 mM dNTP, 4 μl of 0.1 M DTT, 2 μl of 40 U/μl RNase inhibitor, and 8 μl of a 5× first strand buffer were added. After the solution was warmed at 42° C., 2 μl of Superscript II was added, followed by 50 minutes of reaction at 42° C. This reaction solution was used as a template for the next PCR reaction. PCR reaction was performed using the VIP36 gene 5′ terminal and 3′ terminal oligo DNA primers (Example 1, VIPran1, 5) used upon library construction. Reaction was conducted using KOD-plus DNA polymerase (TOYOBO) under conditions of 35 cycles of denaturation at 94° C. for 15 seconds, annealing at 51° C. for 30 seconds, and elongation at 68° C. for 1 minute. By agarose electrophoresis, a band corresponding to approximately 960 nucleotide pairs was confirmed and the thus obtained DNA fragment was collected. The collected gene was inserted into the Sma I site of pBluescript II SK, replicated within Escherichia coli, and then extracted. Thus, the nucleotide sequence was determined. As a result of analyzing a gene encoding the altered VIP36 that had been introduced into clone 12, the nucleotide sequence encoding the mutated loop was found to be 5′-GACCCTGATTCTAATGGTGGTTCTTTT-3′ (SEQ ID NO: 16) and predicted amino acid sequence was Asp-Pro-Asp-Ser-Asn-Gly-Gly-Ser-Phe (SEQ ID NO: 17).

All publications, patents, and patent applications cited herein are incorporated herein by reference in their entirety.

### INDUSTRIAL APPLICABILITY

According to the present invention, a method for modifying a carbohydrate moiety and cells expressing glycoproteins having a modified carbohydrate moiety are provided. Such a carbohydrate moiety (oligosaccharide) having a specific sugar structure and glycoproteins having such a carbohydrate moiety are required in the fields of medicine. Since such oligosaccharides and proteins can be produced conveniently and rapidly, the method for modifying a carbohydrate moiety and the cell expressing such glycoproteins having a modified carbohydrate moiety according to the present invention are useful.

Furthermore, the present invention makes it possible to construct a oligosaccharide library comprising cells expressing various oligosaccharides, generate unknown oligosaccharides, and examine the usefulness thereof.

SEQUENCE FREE TEXT

SEQ ID NOS: 5 to 16: synthetic oligonucleotide (N=A, T, G. or C)

SEQ ID NO: 17: synthetic peptide

1-13. (canceled)

14. A eukaryotic cell comprising heterologous DNA coding for a cargo receptor that is characterized by an alteration of its carbohydrate recognition domain, such that said cell expresses a glycoprotein with a modified carbohydrate moiety.

15. A eukaryotic cell according to claim 14, wherein said glycoprotein is a membrane-bound protein or a secretory protein.

16. A eukaryotic cell according to claim 14, wherein said cargo receptor is at least one of VIP36 and ERGIC-53.

17. A plurality of eukaryotic cells according to claim 14, wherein said cells together express a variety of carbohydrate recognition domains of a cargo receptor.

18. A plurality of eukaryotic cells according to claim 17, wherein said plurality is enriched for eukaryotic cells that express glycoprotein characterized by a particular glycoform.

19. A method for producing a glycoprotein, comprising culturing of a eukaryotic cell according to claim 14 and then collecting said glycoprotein from said cell.

20. A method according to claim 19, further comprising cleaving said glycoprotein to release an oligosaccharide.

21. A method for screening for a test substance, comprising bringing said test substance into contact with a eukaryotic cell according to claim 14 and then detecting the presence or absence of an interaction between said test substance and said glycoprotein.


SEQUENCE LISTING (17 sequences)

SEQ ID NO: 1 - DNA, 2768 bp, Homo sapiens
  CDS (22)..(1554)
  sig_peptide (22)..(112)
ggtcgcgttccagaatccaagatggcgggatccaggcaaaggggtctccgggccagagttcggccgctgttctgcgccttgctgctgtcactcggtcgcttcgtccggggcgacggcgtgggaggagaccccgcggtcgcgttgccacatcgccgtttcgagtacaaatacagcttcaaggggccgcacctggtgcagagcgacgggaccgtgcccttctgggcccacgcggggaatgctattccaagttcagatcaaattcgagtagcaccatctttaaaaagccaaagaggctcagtgtggacaaagacaaaagcggcctttgagaactgggaagttgaggtgacatttcgagtgactggaagaggtcgaattggagctgatggcctagcaatttggtatgcagaaaatcaaggcttggagggccctgtgtttggatcagctgatctgtggaatggtgttggaatattttttgatacttttgacaatgatggaaagaaaaataatcctgctatagtaattataggcaacaatggacaaatccattatgaccatcaaaatgacggggctagtcaagctttggcaagttgccagagggacttccgcaacaaaccctatcctgtccgagcaaagattacctattaccagaacacactgacagtaatgatcaataatggctttacaccagataaaaatgattatgaattttgtgccaaagtggaaaatatgattatccctgcacaagggcattttggaatatctgctgcaactggaggtcttgcagatgaccatgatgtcctttcttttctgactttccagttgactgaacctggaaaagagccgcccacaccagataaagaaatttcggaaaaggaaaaagaaaagtatcaggaggaatttgagcactttcaacaagaattggataaaaaaaaagaggaattccagaagggccaccccgacctccaagggcagcctgcggaggaaatatttgagagtgtaggagatcgagagctaagacaagtctttgaaggacagaatcgtattcatcttgaaatcaagcagctgaaccggcagttagatatgattcttgatgaacagagaagatatgtctcttccttaacagaggaaatctctaaaagaggagcaggaatgcctgggcagcatgggcagattactcaacaagaactggatactgttgtgaaaactcagcatgagattctgagacaagtaaatgaaatgaaaaattccatgagtgaaaccgtcagactggtcagtggaatgcagcaccctggctctgctggaggcgtctatgagacaacacagcacttcattgacatcaaagagcacctgcacatagtaaagagggacatagataacttagtgcagcgaaatatgccatcaaatgaaaagccgaaatgcccagaactaccaccatttccatcatgtttgtctacggtccacttcattatatttgttgtggtgcaaactgtattattcattggttatatcatgtataggtctcagcaagaagcagctgccaaaaaattcttttgactaccattttcctgtgtacttcatctatttgtgtacaaaatgagtcgttttgagggaatttaagtatttaaattgcttcatagtctaaattattaattttcttaataaaataactgtttaaacattgatttgcagttaagaataaaccttaaagcaaagacaaccacattttaatttgttcacagtatgtaaatctgtctaaatttcagtgaatttctggtcagtatgatgcagcctctgagcagaatattgaccagtaagagggtaaataaagtgggggcaaccctggatatgaatgttaccccctaagtctccaatattgcaggtttccctgtataacgtaaacacacttgccctcatgcctcccagaatatgaggtctaattaagaagtccatcaggtttattttgtaaccaaagtcttttttagaggtcagacttcctaatcaaaggcctgggcctgcagtccctttcatcttaatgcaacttcctttgaaatcaaagaatattttgtctgagagctttaaggatctggtaatagacttcaaaatgttaagtgaaatttttttttcctctatttatcaatgatatatttcacttttaaaggaaattttagaggaaaattaatagctgctttttgcactaaaaaaccttgtgggtggaaatattcctctgagaatggcttttataggtattttgcctggtaatgtattcattcatgattgcccatattcttgaatgtttcttcattccaatggggtcaggtcaatattatgaaaataatttttatatttatatttgtaactaagaatttatttctccctttactacacgatgtaaattcacgtcaaattcgatgatctgaggatttaaattcacaaaacctgccactacattctggtttacattagttacttcatgctggctggggttagtgaccatttgcatactcttttaaatcaaggaggctgtagtagaggcagttttaagattcttgaaggcaaaatttgaaaaacagtgaatacttctaattgtttccttttagtgccagaactaagacattgtgaagcacttgttagtaaacttaaccttgaaatgtcagactggaaggagtttttatgtctttgtgcatacttctgggtattacagaaacagtctgtaaataacattttaagatgcaaatttaattctgttcacagctgatttatactgattt

SEQ ID NO: 2 - protein, 510 aa, Homo sapiens [sequence omitted]

SEQ ID NO: 3 - DNA, 1407 bp, Homo sapiens
  CDS (1)..(1071)
  sig_peptide (1)..(134)
atggcggcggaaggctggatttggcgttggggctggggccggcggtgcctgggaaggcctgggcttctcggccccggccctggccccactacacctctctttcttcttttgttgttggggtctgtgactgcggatataactgacggcaacagtgaacatctcaagcgggagcattcgctcattaagccctaccaaggggtcggttccagctctatgcccctctgggacttccagggcagcactatgctcacgagccagtacgtacgtctgacccctgacgagcgcagcaaagagggctctatctggaaccaccagccgtgcttcctcaaagactgggaaatgcacgtccacttcaaagtccacggcacagggaagaagaacctccatggagacggcatcgccttgtggtacacccgggaccgcctcgtgccagggcctgtgtttggaagcaaagataacttccacggcttagccatcttcctggacacctaccccaatgatgagaccactgagcgcgtgttcccgtacatctcggtgatggtgaacaatggctccctgtcctacgaccacagcaaggatgggcgctggaccgagctggcgggctgcacggctgacttccgcaaccgcgatcacgacaccttcctggctgtgcgctactcccggggccgtctgacggtgatgaccgacctggaggacaagaacgagtggaagaactgcattgacatcacgggagtgcgcctgcccaccggctactacttcggggcctccgccggcaccggcgacctgtctgacaatcatgacatcatctccatgaagctgttccagctgatggtggagcacacgcccgacgaggagagcatcgactggaccaagatcgagcccagcgtcaacttcctcaagtcgcccaaagacaacgtggacgaccccacggggaacttccgcagcgggcccctgacggggtggcgggtgttcctgctgctgctgtgcgctctcctgggcatcgttgtctgcgccgtggtgggggccgtggtgttccagaagcggcaggagcggaacaagcgcttctactgagtggcgcctccggcggggcctgtccctgggcccaggagccaatgtgaacttttttttttaccgggattataaaagaacaacaagatgaccttatttcttaactgtttcaaataaatgattaaagtattttcatacattttgcttcttgcccagcagggacaggtggcagagccgaggcttagggtctggcaccccccacagctggagacggaggctctcctggggctggtgtctcaggagcaggggtctgtgtctacagatgggctgtggcccctgcaggcagctgttgaacactggagggtcccccggaccacactggggtgggctcctgaggac

SEQ ID NO: 4 - protein, 356 aa, Homo sapiens
MAAEGWIWRWGWGRRCLGRPGLLGPGPGPTTPLFLLLLLGSVTADITDGNSEHLKREHSLIKPYQGVGSSSMPLWDFQGSTMLTSQYVRLTPDERSKEGSIWNHQPCFLKDWEMHVHFKVHGTGKKNLHGDGIALWYTRDRLVPGPVFGSKDNFHGLAIFLDTYPNDETTERVFPYISVMVNNGSLSYDHSKDGRWTELAGCTADFRNRDHDTFLAVRYSRGRLTVMTDLEDKNEWKNCIDITGVRLPTGYYFGASAGTGDLSDNHDIISMKLFQLMVEHTPDEESIDWTKIEPSVNFLKSPKDNVDDPTGNFRSGPLTGWRVFLLLLCALLGIVVCAVVGAVVFQKRQERNKRFY

SEQ ID NO: 5 - DNA, 29 bp, Artificial Sequence
  Description of Artificial Sequence synthetic oligonucleotide
gcatgtcgacataactgacggcaacagtg

SEQ ID NO: 6 - DNA, 30 bp, Artificial Sequence
  Description of Artificial Sequence synthetic oligonucleotide
gagctctagaaagatggctaagccgtggaa

SEQ ID NO: 7 - DNA, 53 bp, Artificial Sequence
  Description of Artificial Sequence synthetic oligonucleotide
cgtgctctagacnnknnknnkaatnnknnknnknnkgagcgcgtgttcccgta

SEQ ID NO: 8 - DNA, 30 bp, Artificial Sequence
  Description of Artificial Sequence synthetic oligonucleotide
atcgtcttaagcactcagtagaagcgcttg

SEQ ID NO: 9 - DNA, 24 bp, Artificial Sequence
  Description of Artificial Sequence synthetic oligonucleotide
gactacaaagacgatgacgacaag

SEQ ID NO: 10 - DNA, 24 bp, Artificial Sequence
  Description of Artificial Sequence synthetic oligonucleotide
ctagaagtccttaagagtcgggcc

SEQ ID NO: 11 - DNA, 16 bp, Artificial Sequence
  Description of Artificial Sequence synthetic oligonucleotide
cgactcttaaggactt

SEQ ID NO: 12 - DNA, 23 bp, Artificial Sequence
  Description of Artificial Sequence synthetic oligonucleotide
gtacgtcgacggcgtgggaggag

SEQ ID NO: 13 - DNA, 30 bp, Artificial Sequence
  Description of Artificial Sequence synthetic oligonucleotide
cgtatctagaaatattccaacaccattcca

SEQ ID NO: 14 - DNA, 58 bp, Artificial Sequence
  Description of Artificial Sequence synthetic oligonucleotide
cgtatctagatnnknnknnkaatnnknnknnknnkaataatcctgctatagtaattat

SEQ ID NO: 15 - DNA, 32 bp, Artificial Sequence
  Description of Artificial Sequence synthetic oligonucleotide
cgtacttaagtggtagtcaaaagaattttttg

SEQ ID NO: 16 - DNA, 27 bp, Artificial Sequence
  Description of Artificial Sequence synthetic oligonucleotide
gaccctgattctaatggtggttctttt

SEQ ID NO: 17 - protein, 9 aa, Artificial Sequence
  Description of Artificial Sequence synthetic peptide
DPDSNGGSF